(19) United States
(12) Patent Application Publication
(10) Pub. No.: US 2022/0112480 A1
(43) Pub. Date: Apr. 14, 2022

(54) OSCILLATORY METHOD AND DEVICE FOR REDUCING BACTERIA, VIRUSES AND CANCEROUS CELLS

(71) Applicant: John P Keady, Fairfax Station, VA (US)

(72) Inventor: John P Keady, Fairfax Station, VA (US)

(21) Appl. No.: 17/185,957

(22) Filed: Feb. 25, 2021

Related U.S. Application Data
(63) Continuation-in-part of application No. 15/922,867, filed on Mar. 15, 2018, now abandoned.
(60) Provisional application No. 63/015,388, filed on Apr. 24, 2020.
(60) Provisional application No. 62/471,861, filed on Mar. 15, 2017.

(51) Int. Cl. C12N 13/00; A61H 23/02; G01H 13/00

(52) U.S. Cl. CPC C12N 13/00; G01H 13/00; A61H 23/0245; A61H 23/0236

(57) ABSTRACT

At least one embodiment is directed to a method reducing the growth of a pathogen by targeting the pathogen by a vibrational wave at an integer fraction of its fundamental frequency, at low amplitudes so as to not harm healthy tissue, for a minimal exposure time determined by wave amplitude and damping.

### CROSS-REFERENCE TO RELATED APPLICATIONS

This application is a continuation in part of U.S. patent application Ser. No. 15/922,867 filed 15 Mar. 2018, which claims priority benefit of both U.S. provisional patent application No. 62/615,152 filed 9 Jan. 2018 and U.S. provisional patent application No. 62/471,861 filed 15 Mar. 2017. This application additionally is a nonprovisional application that claims priority to U.S. provisional patent application No. 63/015,388, filed 24 Apr. 2020. The disclosures of which are all incorporated herein by reference in their entirety.

### FIELD OF THE INVENTION

The present invention relates to devices that can be used to generate oscillations that can be used to disrupt bacteria, viruses, and cancerous cells.

### BACKGROUND OF THE INVENTION

Viruses include a genome and often enzymes encapsulated by protein capsid, with often a lipid envelope. A virus must subjugate a host to reproduce, and various methods are used to attack viruses throughout their life cycle. Two common methods used are vaccines and anti-viral drugs. Vaccines can be effective on stable viruses but not on infected patients or fast mutating viruses. Anti-viral drugs target viral proteins. The disadvantage of anti-viral drugs is the eventual pathogen mutation over time and the hazard of side effects if the viral proteins are similar to human proteins.

The market for anti-viral drugs totals in the billions of dollars. Generics in global antivirals market are estimated to be $4.2 billion in 2010 and are forecast to reach $9.2 billion by 2018. Generics in the HIV market accounted for 46% of market share in total generic antivirals market in 2010, while generic herpes therapeutics accounted for 39.6% of market share. Generic influenza therapeutics accounted for 1% of total market share.

It has been reported that in 2002, the annual treatment for HIV/AIDS cost an average of $9,971. This grew substantially at a compound average growth rate (CAGR) of 3.2% to $12,829 in 2010. Deaths in 2011 as a result from HIV/AIDS was greater than 1 million worldwide.

A method of permanent viral eradication, without drugs, without the possibility of pathogen mutation, and with equipment that can treat patients in few visits, would save millions of lives and billions of dollars each year. Additionally the technique could be applied to sterilizing medical instruments, bacteria and some cancers.

### BRIEF DESCRIPTION OF THE DRAWINGS

Exemplary embodiments of present invention will become more fully understood from the detailed description and the accompanying drawings, wherein:

FIG. 1 illustrates a pulse wave upon a pathogen;

FIG. 2 illustrates generating a pulse wave upon a pathogen;

FIG. 3 illustrates the deformation of a pathogen from a pulse wave;

FIG. 4 illustrates a linear model of deformation;

FIG. 5 illustrates the amplitude effect of a less than the fundamental frequency driving wave;

FIG. 6 illustrates the amplitude effect of a larger than fundamental frequency driving wave;

FIG. 7 illustrates the amplitude effect of a near fundamental frequency driving wave;

FIG. 8 illustrates generating a pulse wave upon a pathogen;

FIG. 9 illustrates the deformation of a pathogen form a pulse wave;

FIG. 10 illustrates linear models of radial deformation;

FIGS. 11A, 11B, and 11C illustrate an atomic force microscope (AFM) image of a virus (11A), that has been damage (11B), and the related force causing deformation from the AFM cantilever system (11C);

FIG. 12 is a table of Young's modulus of E. coli;

FIG. 13 is a table of density of E. coli;

FIG. 14 illustrates a linear deformation model;

FIG. 15 illustrates a radial deformation model;

FIGS. 16 and 17 illustrate optical density (OD) curves;

FIGS. 18 and 19 illustrate optical density (OD) ratio curves;

FIGS. 20 and 21 illustrate an OD curve and it's related OD ratio curve respectively;

FIGS. 22, 23, 24A, 24B, 24C, 24D, 25A, 25B, 25C, 25D, 26A, 26B, 26C, 26D, 27 illustrate bins of the bin model for bacterial replication;

FIGS. 28 and 29 illustrate a simulated OD curve and it's related OD ratio curve respectively;

FIGS. 30 and 31 illustrate a simulated OD curve and it's related OD ratio curve respectively;

FIGS. 32 and 33 illustrate a simulated OD curve and it's related OD ratio curve respectively;

FIG. 34 illustrates a simulated OD curve;

FIG. 35 illustrates a simulated OD curve;

FIG. 36 illustrates a simulated OD ratio curve;

FIGS. 37 and 38 illustrate models of bacteria;

FIGS. 39 A and 39 B illustrates aspects of the E. coli bacteria;

FIG. 40 illustrates a model of a bacteria;

FIG. 41 illustrates acoustic emission data;

FIG. 42 illustrates a table of modelling values for E. coli;

FIG. 43 illustrates a table of modelling values for E. coli;

FIG. 44 illustrates a non limiting example of an acoustic source;

FIG. 45 illustrates the experimental setup to test proof of concept on E. coli;

FIGS. 46, 47 and 48 illustrate the experimental test setup for testing surface viability of a healthy cell exposed to target acoustic waves;

FIGS. 49A and 49B illustrate test results of cell function when exposed to target acoustic waves;

FIGS. 50, 51, 52, 53, 54, 55, 56, 57, 58, 59 and 60 illustrate proof of concept experimental results;

FIG. 61 illustrates a table that shows modulus values for cancerous cells compared to healthy cells;

FIG. 62 illustrates a method of measuring elastic properties of a bacteria using an AFM;

FIGS. 63, 64, 65, 66, 67, 68, 69, 70, 71, 72, 73, and 74 illustrate various exemplary embodiments in delivering the acoustic waves to a pathogen or determining the resonant frequency of the pathogen;

FIG. 75 illustrates a method of creating an oscillating AFM platform;

FIGS. 76 and 77 illustrate an AFM needle on an oscillating platform and the measurement taken;

FIG. 78 illustrates a method to obtain the resonance frequency or integer fraction of the resonance of a pathogen directly;

FIG. 79 illustrates the result of sweeping AFM platform oscillation frequency and measuring the amplitude that makes it to the AFM probe;

FIG. 80 illustrates a modified result of sweeping AFM platform oscillation frequency and measuring the amplitude that makes it to the AFM probe;

FIG. 81 illustrates a modified result of sweeping AFM platform oscillation frequency and measuring the amplitude that makes it to the AFM probe across multiple integer fractions of the resonance frequency;

FIG. 82 illustrates a water method for delivering the acoustic frequencies necessary for body treatment in a prone position;

FIG. 83 illustrates a water method for delivering the acoustic frequencies necessary for body treatment in a sitting position;

FIG. 84 illustrates a finger method of analysis and treatment;

FIG. 85 illustrates prone method of analysis and treatment;

FIG. 86 illustrates a cup incorporating analysis and treatment method for pathogen eradication;

FIG. 87 illustrates a cross-section of a cup incorporating analysis and treatment method for pathogen eradication;

FIG. 88 illustrates a cross-section of an analysis and treatment sampling cup;

FIG. 89 illustrates a cross-section of an analysis and treatment sampling cup with the inserted sample;

FIG. 90 illustrates an analysis and treatment sampling cup with the inserted sample;

FIGS. 91, 92, and 93 illustrate several views of a biometric sensor bracelet that can additionally analyze and treat pathogens;

FIGS. 94, 95, 96, and 97 illustrate several views of a biometric sensor ring that can additionally analyze and treat pathogens; and

FIG. 98 illustrates a method of analysis and treatment using inserted acoustic detectors and emitters.

### DETAILED DESCRIPTION OF EMBODIMENTS

The following description of exemplary embodiment(s) is merely illustrative in nature and is in no way intended to limit the invention, its application, or uses.

Atomic force microscope studies on viruses, in order to avoid destruction of a virion during imaging, have determined that a deformation between 20-30% of the diameter of the virion results in rupture of the capsid, whether the capsid is empty or full of genomic material (Nurmemmedov et al., Biophysics of viral infectivity: matching genome length with capsid size, Quarterly Reviews of Biophysics, 40, p. 327-356, 4 (2007)).

Unlike the motivation of the Nurmemmedov et. al study, the motivation for the invention was to render viruses, bacteria, and certain cancer cells benign by disrupting the surface or internal structure of the pathogens (note cancer is included under this term as is viruses and bacteria and any other type of non-normal structure in a biological unit (e.g., human)) to a point where the replication of the pathogen is disrupted. Instead of direct complete deformation (i.e., target deformation due to initial pulses, such as occur in high acoustic amplitude ultrasonic cleaners, which also damage healthy tissue), exemplary embodiments seek to reach disruption deformation values by gradual applied oscillations at integer fractions of the natural resonance of the pathogen, which is unique compared to healthy tissue. A gradual applied (e.g., lower amplitude over a longer period of time) amplitude protects healthy tissue but focuses on specific target frequencies to disrupt pathogens. This is contrary to typical ultrasonic sterilization device that uses high amplitudes to destroy all tissue, or ultrasonics that are used for diagnostics at set frequencies that are not tailored for specific pathogens but for ease of analysis. In at least embodiment of the present invention the size of the pulse will determine whether the deformation oscillation is considered a linear oscillation (pulse width<pathogen size) or radial oscillation (pulse width>pathogen size). Several exemplary embodiments are directed to determining the resonant frequency needed and then apply integer fractions of the resonant frequencies to disrupt the surface structure and/or interior of the pathogen. Additional exemplary embodiments provide methods and devices to determine resonant frequencies, and deliver the pulses at integer fractions. The advantage of the present invention is a disruption device and method that a pathogen can not mutate to avoid. In at least one further embodiment the treatment frequency, amplitude and waveform (e.g., sinusoidal, pulse, non-linear) can be temporally altered during the treatment time. For example, to take into account damping, the amplitude can be gradually increased over time, always being below the amplitude that would cause tissue harm on initial contact.

### Properties of Resonance

Any homogenous object and even inhomogeneous objects that have a well-defined size, shape, and/or material property, will have an inherent natural frequency of structural oscillation. Depending upon the damping characteristics, an internal oscillation, driven by an external source at the natural frequency, can result in an internal energy increase that could exceed the molecular bonding energy of the object, at which point the object integrity is compromised. Additionally, in many cases it is not necessary to break molecular bonding but instead to disrupt surface structure such that the pathogen can not bond with a host. For example, an external driving source can initiate, for example by an oscillating pressure pulse on a contact portion of the object, an internal travelling pulse. The internal pulse will travel according to the object properties such as density and bulk modulus, reflecting at the end of the object boundary back to the position of the original contact point. The remaining energy in the reflecting internal pulse, upon reaching the contact portion, is dependent upon the damping properties of the object. If the reflected internal pulse is not fully damped then any subsequent external pressure pulse at a characteristic frequency unique to the object will build upon any subsequent internally reflected pulse. If the subsequently built upon internal pulses have energies greater than the bonding energy of the object, deformation and subsequent rupture can occur. The internal oscillations will additionally depend upon the characteristics of the external oscillating pressure pulse. For example, if the externally driven pulse widths are greater than a characteristic dimension of the object then the object may experience radial contraction and expansion, modeled in two or three dimensions. If the pulse width is smaller than a characteristic dimension of the object a longitudinal contraction and expansion may be experienced, and hence can be one dimensionally modeled.

FIG. 2 illustrates a simplified representation of a pathogen/particle being impinged by an external driving pulse having a pulse width λ less than a characteristic dimension ‘d’, referred herein as the first pulse condition. Condition λ>d is referred to as the second pulse condition. The speed of the internal deformation is c1 and the speed of the external pressure pulse is c0. Often c1>c0, for example for 20° C. water c0 is about 1481 m/sec, while for glass c1 is about 3960 m/sec. Very few references cite the speed of sound in a virus/bacterium, but at least one reference cites the range between 900-1800 m/sec. (Tsen et al., 2007) The medium (e.g. aqueous solution) through which the external pulse travels is about c0=1481 m/sec. A range of 900-1800 m/sec for a target virus results in two relative speed conditions to examine, one where c₁<c₀ and the other where c₁>c₀. When c1<c0, then the exerted external pressure pulse will be felt by the virus/particle as an impulse. If c1>c0 then the exerted external pressure pulse will drive the virus/particle oscillation irrespective of the natural frequency. If the drive frequency can match the natural frequency, then disruption can occur. It is important to note at this point that it is not the intention of this study to attempt to ‘shatter’ objects by resonance, although that is one possible outcome, but the goal is to deform the object to a level that disrupts its ability to attach to a host and/or replicate.

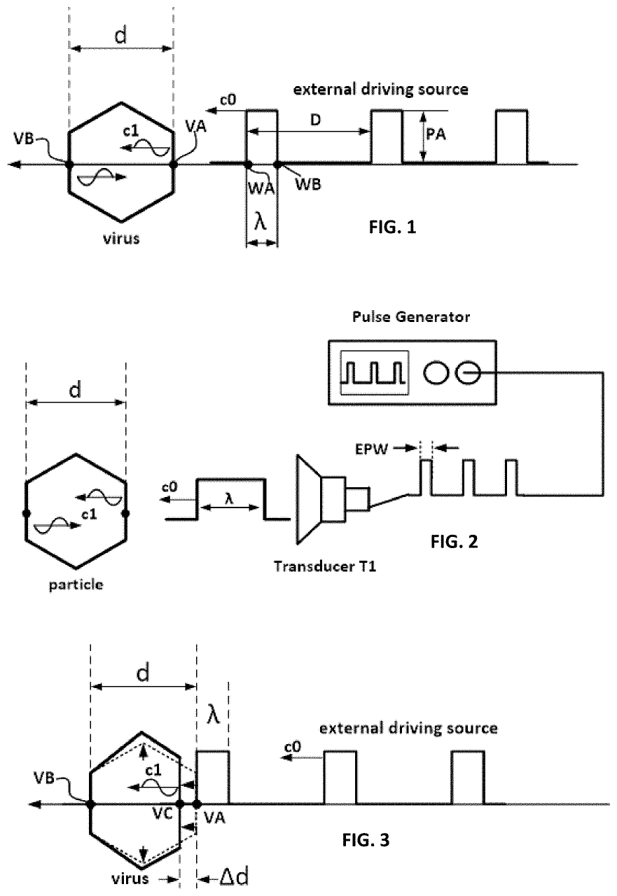

Referring to FIG. 1 where an externally driven pulse width λ<d (dimension of pathogen), the resonance frequency, ‘fᵣ’, is related to the time an internal pulse, starting at position VA, reflects at position VB and returns to position VA. The time of travel is dependent on the internal speed, c₁, and the distance travelled ‘2d’, where the resonance frequency, ‘fᵣ’, in Hz can be expressed as:

$${f}_{r} = \frac{{c}_{1}}{2 d} \qquad ( 1 )$$

If the internal damping allows multiple internal reflections, ‘n’, prior to another external pulse, then a lower pulse frequency can be used to build up the critical destructive energy level. The lower pulse frequencies (number of pulses/sec) can be expressed as:

$${f}_{nr} = \frac{{c}_{1}}{n 2 d} \qquad ( 2 )$$

Equation 2 specifies a natural frequency (n=1) and subsequent frequencies that are integer fractions of the natural frequency. These frequencies can be initiated by external driving forces (e.g. external acoustic pulses) and internal driving forces (e.g., natural bacterial metabolism and growth mechanisms).

For small sizes typically the natural frequency is in the GHz range. For example, if c₁=1200 m/sec, d=100 nm, then f₁ᵣ=6 GHz, which would be the external pulse frequency needed if internal damping is large (near critical damping) where multiple internal reflections do not occur. If the damping is low such that 100 reflections can occur prior to another external pulse hit, then with n=100, the resonance frequency would be f₁₀₀ᵣ=60 MHz. Current technology used in computer engineering, for example computational bit generation, can generate pulses on the order of 20 GHz-60 GHz, however generating acoustic pulses is limited to the highest MHz value that available immersion transducers can generate, as large as 225 MHz (e.g., Olympus high frequency flaw detectors), however it is anticipated that greater than 225 MHz generators will become available. An additional consideration is whether the electrical pulse width that drives a transducer produces the desired acoustic pulse, for example one that has a pulse width λ less than ‘d’.

FIG. 2 illustrates pulse generator driving a pulse (acoustic or electromagnetic) the relationship between the electric pulse width EPW driving a transducer T1, and acoustic pulse of acoustic pulse width λ. To determine the electrical pulse width EPW needed, usually expressed in terms of time, the EPW must be translated into acoustic pulse width. For example, if the electrical pulse width is 20 nsec this translates into a transducer flexing for about 20 nsec but typically longer due to mechanical response of the transducer. The acoustic pulse width λ associated with the 20 nsec transducer flex translates to 20 nsec×1481 m/sec=29620 nm or 29.62 microns. Thus the ideal relation between electrical pulse width EPW, to acoustic pulse width λ can be expressed as:

λ=EPW*c₀ (3)

Where c0 is the speed of sound in the medium in which the transducer is immersed. Therefore, to get an acoustic pulse A on the order of 100 nm then EPW=0.675 psec, which generally relates to a 14.8 GHz pulse generator. Current technology is pushing 60 GHz, which relates to an acoustic pulse width of 24.6 nm. If the internal damping is low and ‘n’ large then the pulse frequency can be much lower even if the pulse width is the same. For example, in the above example where f₁ᵣ=6 GHz, a 60 GHz electrical pulse generator can be used to generate a 24.6 nm acoustic pulse width at a lower frequency than 60 GHz for example in this case 6 GHz. If ‘n=100’, or the damping is such that 100 reflections can occur before the next external pulse, as discussed above, then the 24.6 nm acoustic pulse width can be generated at a pulse frequency of 60 MHz (an integer fraction of the resonance), well within the 225 MHz upper limit of immersion transducers.

FIG. 1 illustrated, the first pulse condition, where an acoustic pulse width λ is less than the particles dimension ‘d’. FIG. 3 illustrates a linear deformation occurring when the pulse width λ is less than the characteristic dimension d. Although the models developed herein are two dimensional models with multiple springs, FIG. 3 is illustrative of linear deformation.

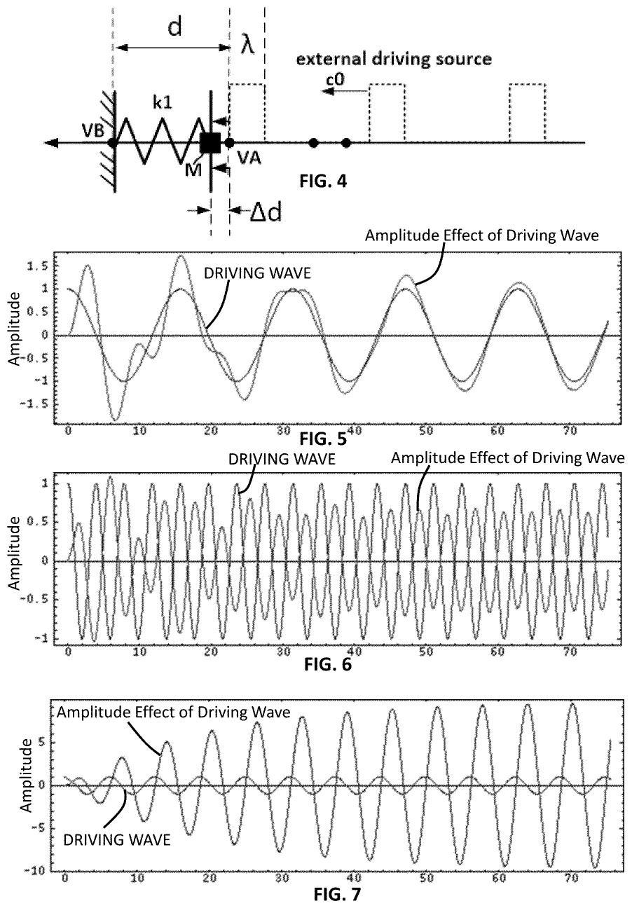

The generalized deformation illustrated in FIG. 3 can be modeled as a simple driven harmonic oscillator. FIG. 4 illustrates a spring model of the linear deformation of FIG. 3. The frequency of oscillation fₒ for the simple harmonic system can be expressed as:

$$2 π {f}_{o} = \sqrt{\frac{k}{m}} = {ω}_{0} \qquad ( 4 )$$

If the VB position is chosen as the origin of a linear x-axis with positive values extending to the right the generalized equation of undamped motion of the mass M can be expressed as:

$$M \frac{{d}^{2} \vec{x}}{d {t}^{2}} + {k}_{1} \vec{x} = \vec{F} ( t ) \qquad ( 5 )$$

Where F(t) is the time dependent driving force. If the driving force is at a frequency less than the resonance frequency the amplitude of the position of the mass M stabilizes. For example, FIG. 5 illustrates the case where the driving force (black line) is sinusoidal at a frequency that is 0.4 f₀ and the amplitude of the sinusoidal solution of the position (purple line) of the mass M stabilizes. (http://www.acs.psu.edu/drussell/Demos/SHO/mass-force.html)

If the driving force is at a frequency greater than the resonance frequency the amplitude of the position of the mass M is less than the driving force. For example, FIG. 6 illustrates the case where the driving force (black line) is sinusoidal at a frequency that is 1.6 f₀ and the amplitude of the sinusoidal solution of the position (red line) of the mass M is less than the amplitude of the driving force. (http://www.acs.psu.edu/drussell/Demos/SHO/mass-force.html)

If the driving force varies in time at a frequency that is some integer fraction the harmonic/resonant frequency of the system (eqn. 4) the value of the position of the mass M will continue to increase bounded by the material constraints of the system. For example FIG. 7 illustrates the case where the driving force (black line) is sinusoidal at a frequency that is 1.01 f₀ and the amplitude of the sinusoidal solution of the position (blue line) of the mass M increases. (http://www.acs.psu.edu/drussell/Demos/SHO/mass-force.html)

If the system is damped, where an initial oscillation is damped by a damping factor ‘c’, the general equation of motion in one dimension can be expressed as:

$$M \frac{{d}^{2} \vec{x}}{d {t}^{2}} + c \frac{d \vec{x}}{d t} + {k}_{1} \vec{x} = \vec{F} ( t ) \qquad ( 6 )$$

The characteristic frequency, including damping ‘c’, can be expressed as:

$${ω}^{′} = \sqrt{\frac{k}{M} - \frac{{c}^{2}}{4 {M}^{2}}} \qquad ( 7 )$$

If the driving frequency is close to ω′ the amplitude of the position of the mass M can continue to increase and be large if the damping is small. For example, if c=0, the amplitude as a result of a driving frequency close to resonant frequency can be theoretically infinitely large, however practically limited by material constraints. If the driving force is a circular function, for example cos(ωt), the equation of motion can be expressed as:

$$M \frac{{d}^{2} x}{d {t}^{2}} + c \frac{d x}{d t} + {k}_{1} x = {F}_{0} cos ( ω t ) \qquad ( 8 )$$

The analytical solution can be expressed as:

x=A₀ sin(ωt+φ₀) (9)

Where A₀ is the amplitude (maximum position of the mass with respect to a reference point) and can be expressed as:

$$\begin{matrix}{A}_{0} = \frac{{F}_{0}}{M \sqrt{{( {ω}^{2} - {ω}_{0}^{2} )}^{2} + ( {c}^{2} {ω}^{2} / {M}^{2} )}} & ( 10 ) \\ {φ}_{0} = {tan}^{- 1} \frac{{ω}_{0}^{2} - {ω}^{2}}{ω ( c / M )} & ( 11 )\end{matrix}$$

If ω=ω₀ then eqn. 10 can be expressed as:

$${A}_{0} = \frac{{F}_{0}}{M \sqrt{( {c}^{2} {ω}^{2} / {M}^{2} )}} = \frac{{F}_{0}}{c ω} \qquad ( 12 )$$

We can rewrite eqn. (12) in terms of F₀ as:

$$\frac{{A}_{0}}{{F}_{0}} = \frac{1}{c ω} \qquad ( 13 )$$

The damping, if not critically damped, will slow the rate of resonant increase of FIG. 7, however the amplitude will continue to increase.

For a particular particle (e.g., polyethylene sphere, bacteria, virus, cancer cell) the various characteristics of the harmonic model, for example the equivalent mass, M, and the equivalent spring constant, k, may not be known, and must be either modelled or obtained by acoustically ‘pinging’ the pathogen and measuring acoustic emissions. The frequency of oscillation for a simple harmonic oscillator can be rewritten in terms of a single unknown, s=k/m.

fₒ=2π\sqrt{s} (14)

One method to determine ‘s’, is to impart a pulse from an external source and measure any resonance acoustic emissions. As discussed below when reviewing bacteria properties, researchers have detected acoustic emissions (AE) from bacteria during growth. Although the AE from bacterial growth is not in response to an external pulse (ping), we argue herein that it is due to internal driving forces. The resonance emissions should occur at f₀ or some integer fraction, providing experimental information as to which frequencies to use for resonant disruption as well as a characteristic ‘s’ value for a particular particle. It will also provide information on damping by measuring emitted resonant peak reduction over time following an initial pulse trigger (either originating internal or external). Note that herein is discussed externally initiated resonances in particles/bacteria/viruses/cells, however internal forces originating for example from growth, could also trigger oscillations where the resonant values are less damped than other values and manifest themselves as AEs.

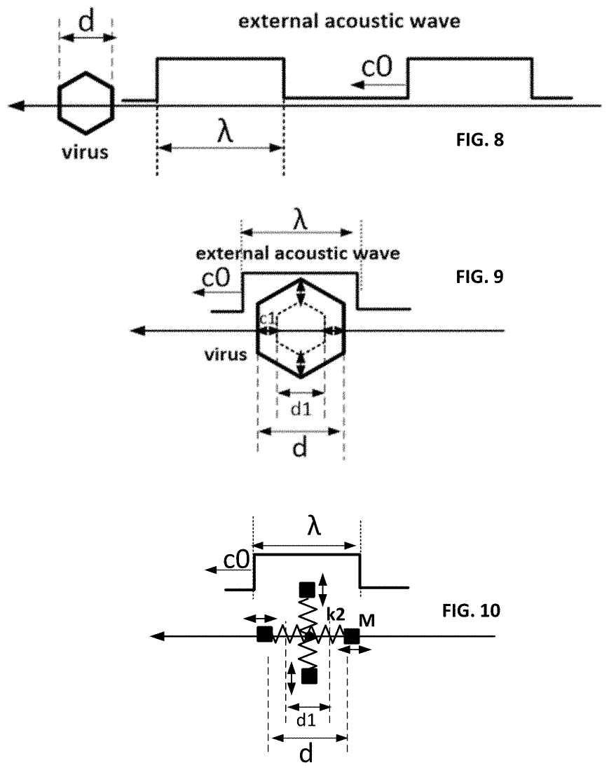

FIGS. 8, 9, and 10 represent the equivalent to FIGS. 1, 3, and 4 respectively for the second pulse condition where the pulse width is larger than dimension ‘d’.

When the pulse width ‘λ’ is larger than dimension ‘d’, the external impulse can be viewed as a compression-expansion of the entire surface of the particle, which can be modeled as a radial oscillation with respect to a central point as shown in FIGS. 9 and 10.

FIG. 9 illustrates a radial deformation occurring when the pulse width λ is greater than the characteristic dimension d. FIG. 10 illustrates a spring model of the 2-D deformation of FIG. 8. If the internal pulse speed ‘c₁’ is greater than the external pulse speed ‘c₀’, then since all of the surface is exposed to the compression-expansion driving pulse, any internal oscillation may oscillate about the imposed surface compression or expansion. If the internal pulse speed ‘c1’ is less than the external pulse speed ‘c₀’, then a multiple of reflections can occur before another external pulse is encountered, as well as enabling the imposition of resonance.

The concept behind at least one exemplary embodiment was tested on a pathogen, E. coli. A non-limiting example of a model of the E. coli bacteria was also developed. In the models a simplified spring model and spring constant, k, are derived using material properties obtained from the literature for example from atomic force microscope studies, discussed below. The driving force is provided by acoustic pulses. Models developed herein are only nonlimiting examples and are dependent upon the physical properties of a particle/pathogen, such as mass, density, elasticity, and resonant modes. In the example herein acoustic emission data reported for E. coli was used in models to estimate target frequencies.

### Particle/Pathogen Characteristics

Critical in modeling the resonance frequencies of the target pathogen is obtaining predictions of 1.) internal oscillation speeds; 2.) damping characteristics; 3.) equivalent spring constant kᵥ and 4.) deformation amount needed for surface disruption of the target pathogen. The internal oscillation speed, damping, and spring constant are needed to determine which pulse frequency to expose the pathogen to, while the damping and deformation amount is needed to determine the extent of the exposure time needed to affect infectivity.

Virus Characteristics

Little direct information is available to provide internal oscillation speeds and damping characteristics of viruses, however some information can be obtained by force microscope studies and some molecular modeling studies, which are described below.

As an example, we look at the Rift Valley Fever Virus (RVFV) which is a virus that is commonly used in studies. RVFV is a mosquito-borne virus that infects animals and the Humans that come into contact with infected animal tissue. (Grobbelaar et al., 2011). The average size is about 95 nm+/−9 nm. The diameter is about 96 nm with a standard deviation of 4 nm (Freiberg et al., 2008). The virus includes a shell (capsid) of thickness about 5 nm, with a separation between the capsid and interior core by about 2 nm (Sherman et al., 2009). RFVF is a part of the genus phlebovirus, whose buoyant density is listed as 1.20-1.21 g/cc. (Tidona et al., 2011). Very few references cite the actual density of RVFV, however several older references do measure densities of various viruses. The papilloma virus, with a radius of 33 nm, has a measured density of 1.133 g/cc. (Sharp et al., 1946). The Influenza virus A, B, and Swine, have diameters respectively of 101 nm, 123 nm, and 96.5 nm. The densities of the Influenza virus A, B, and Swine, have measured density values of 1.104 g/cc, 1.104 g/cc, and 1.100 g/cc respectively (Sharp et al., 1945).

Parvovirus has a density of about 1.39 g/cc with a 24 nm diameter, and the hepatitis A virus has a density of 1.34 g/cc with a 29 nm diameter. (Gunter Siegl et al., 1978). The bovine diarrhea virus has a density from 1.09 to 1.15 g/cc, the hog cholera virus has a density from 1.12 g/cc to 1.16 g/cc and the flavivirus has a density from 1.19 g/cc to 1.20 g/cc. (Hideaki Miyamoto et al., 1992). The mass of an individual virus depends on both density and volume, and hence can vary. For example, a vaccinia virus of the Poxviridae family can range in mass from about 5-9.5 fg (10⁻¹⁵ g) (Gupta et al., 2004), while an Influenza A virus particle has a mass of about 5.2×10⁻¹⁶ g (Vollmer et al., 2008). These properties (e.g., density, size, vibrational speeds, mass, resonance frequencies) can be used to model the pathogen (e.g., virus, bacteria, cancerous cells) to obtain target frequencies (e.g., near integer fractions of resonant frequencies). Note that exact integer fraction of resonant frequencies are not needed, but near integer fractions of resonances can be used. For example, a value of f₀/n where ‘n’ can be from 1.8 to 2.2, when the target is n=2.

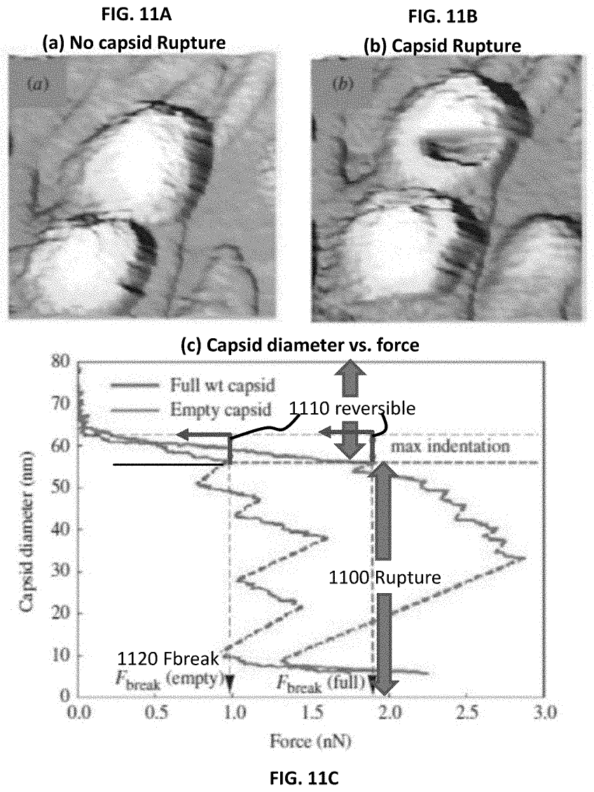

At least one exemplary embodiment is directed to disrupting the viral capsid affecting a pathogen's (e.g., viron's) infectivity (e.g., the ability to replicate or infect a host cell). Several references support that a change in the capsid can affect infectivity, for example subtle changes in the capsid, for example charge and surface structure, can abolish infectivity of HIV (density reported 1.16 g/cc to 1/17 g/cc). (Leschonsky et al., 2007) Viral capsids are typically held together in non-covalent bonding interactions. Atomic Force Microscope (AFM) measurements have been used to study several viral capsid properties. The AFM tip can be applied to a single virion and obtains real-time force-distance curves as the nano meter sized tip is moved to scan the virion surface. AFM force-distance curves are generally broken into two regions, one where the application of a force is reversible (FIG. 11a) and another where the application of the force results in rupture of the viral capsid (FIG. 11b). An example of the two regions can be viewed in FIG. 11c.

The threshold force, onset of the second non-linear region, provides information about the strength of the capsomer-capsomer bond. In the first linear region an effective spring constant, kᵥ, of the capsid surface is obtained, and using a thin shell model Young's Modulus E can be obtained. kᵥ is dependent upon the material and the geometry, while E is a geometry independent material property. To retain internal DNA, a viral capsid must retain up to several atm of internal pressure, with the strength directly related to how much DNA can be internally packed. A deformation between 20-30% of the diameter of the virion results in rupture of the capsid, whether the capsid is empty or full of genomic material. Capsid walls are of similar thickness for most viruses (2-4 nm), with lateral stress (e.g., of an elastic shell) related to the thickness. As the capsid radius is increased the capsid walls generally become thinner and weaker (Nurmemmedov et al., 2007). An oscillation can increase/decrease the dimension in one dimension, eventually weakening the capsid surface to the point of rupture, or disrupting attachment sites on the capsid for attaching the virus to a host cell, rendering the virus unable to infect a host cell. Likewise, a pathogen's (e.g., bacteria and cancer) surface can also be disrupted affecting its replication.

The threshold force (1120 F_{break}, FIG. 11c) varies from virus to virus, for example: an empty λ capsid has a threshold force of 0.8 nN and a kᵥ=0.13 N/m; for a CCMV 0.6 nN; and for the φ29 procapsid 1.5 nN and a kᵥ=0.31 N/m. The properties of viral capsids also change during maturation, for example an immature HIV particle has a Young's Modulus (E) of about 930 MPa while a mature HIV particle has a value of 115 MPa. (Nurmemmedov et al., 2007).

To relate the break force (F_{break}, FIG. 11c) to a pressure one must relate the break force to the area throughout which the force is exerted. For example, a tip is generally used to exert a force, where the force is exerted over a tip area. In general, if the tip has a radius of ‘r’ then the contact area, A_{c}, is approximately:

A_{c}=πr² (15)

Although the tip is generally a three-dimensional cone, eqn. 15 provides a cross sectional area of the cone of contact and underestimates the contact surface area. Thus, the force derived using eqn. 15 will be generally larger than needed. Using the area associated with eqn. 15, a pressure associated with the break/deformation force F_{break} would be:

$${P}_{b} = ( \frac{{F}_{b r e a k}}{π {r}^{2}} ) \qquad ( 16 )$$

For example, a deformation of a capsid of about 20-30% of the diameter, was studied with break forces of F_{break}=2.8 nN for the φ29 capsid over a 20 nm radius tip, providing a P_{b-φ29}=2,229,299 N/m². For the plant virus CCMV, F_{break}=0.6 nN, providing a P_{b-CCMV}=477,707 N/m² (Roos et al., 2007).

Thus the pressure increase due to resonance must reach P_{b-φ29}=2,229,299 N/m² to affect the φ29 capsid infectivity. The time it takes to reach this target level is directly related to the exposure time. The minimum exposure time, Tₑₓₚ, assuming no damping, is related to the minimum number of pulses, Nₘᵢₙ, needed to reach P_{b}:

$${N}_{min} = \frac{{P}_{b}}{{P}_{p u l s e}} \qquad ( 17 )$$

where Pₚᵤₗₛₑ is the pressure exerted by a single pulse that is translated into the virus/particle. For example, if the pulse has a Pₚᵤₗₛₑ=100 N/m² (about 130 dB air equivalent level and 160 dB in water), then Nₘᵢₙ=22293 pulses for the φ29 capsid, and Nₘᵢₙ=4777 pulses for the CCMV capsid. The Minimum exposure time, Tₘᵢₙ₋ₚᵤₗₛₑ, is related directly to the resonance frequency and damping. If we assume for the CCMV capsid with a speed of sound of about c₁=1200 m/sec, a diameter of d=30 nm, then we obtain a resonance frequency of f₁ᵣ=2 GHz (eqn 2). To obtain the pressure amplitude required we will want to expose the CCMV capsid to a pulse frequency for a period of time. If for CCMV we need Nₘᵢₙ=4777 pulses, then assuming no damping the minimum exposure time, Tₘᵢₙ₋ₑₓₚ, can be expressed as:

$${T}_{min \text{-} exp \text{-} undamped} = \frac{{N}_{min}}{{f}_{1 r}} \qquad ( 18 )$$

where for CCMV, T_{min-exp-undamped}=2.4×10⁻⁶ sec.

If there is no damping then one option, so that lower pulse frequencies could be use, could be to expose the virus to a resonance mode ‘n’ frequency instead of the fundamental resonance frequency (n=1) since the resonance mode ‘n’ frequency is lower and easier to achieve with the available immersion transducers. For example, a 6 GHz pulse frequency is not possible currently with immersion transducers, however 60 MHz is. Thus if ‘n=100’ then 60 MHz pulse frequency for an undamped system would suffice to affect the infectivity of a virus having a resonant frequency of 6 GHz. The undamped exposure time can be expressed as:

$${T}_{n \text{-} m ode \text{-} exp \text{-} undamped} = \frac{{N}_{min}}{{f}_{n r}} = \frac{{N}_{min}}{( \frac{{c}_{1}}{n 2 d} )} = \frac{n 2 d {N}_{min}}{{c}_{1}} \qquad ( 19 )$$

To this point we have assumed that the system is undamped. A damped system converts part of the initial pulse pressure into heat or vibrational leakage into the environment, so that upon one cycle of reflection a % of the reflected pulse is lost. Assuming the retained fraction is ‘α’ where α<1, then the deformation pressure, ‘P_{b}’, can be related to the pulse pressure ‘Pₚᵤₗₛₑ’ or ‘Pₚ’ after 1 reflection with a Pulse pressure added upon each reflection:

P_{1rfl}=Pₚ+αPₚ (20)

after 2 reflections with a Pulse pressure added upon each reflection:

P_{2rfl}=Pₚ+α(Pₚ+αPₚ)=Pₚ+αPₚ+α²Pₚ (21)

after 3 reflections with a Pulse pressure added upon each reflection:

P_{3rfl}=Pₚ+α(Pₚ+α(Pₚ+αPₚ))=Pₚ+αPₚ+α²Pₚ+α³Pₚ (22)

after N reflections or N+1 pulses as:

P_{(N+1)Pulses}=P_{Nrfl}=Pₚ(1+α+α²+ . . . +α^{N})=Pₚ*Σₙ₌₀^{N}αⁿ (23)

Note that Σₙ₌₀^{N}αⁿ an converges if |α|<1, where we have defined |α|<1, when damped. No damping would be α=1, and a 1% damping would be α=0.99.

If N→∞ then:

$${\sum }_{n = 0}^{N} {α}^{n} = \frac{1}{1 - α} \qquad ( 24 )$$

If we use (24) as an approximation in a damped system, we can say:

$${P}_{( N + 1 ) Pulses} = {P}_{p} * {\sum }_{n = 0}^{N} {α}^{n} \approx {P}_{p} ( \frac{1}{1 - α} ) \qquad ( 25 )$$

For example, if we assume a 1% damping then (23) becomes:

$${P}_{( N + 1 ) Pulses} \approx {P}_{p} ( \frac{1}{1 - α} ) = {P}_{p} ( \frac{1}{1 - 0.99} ) = 100 {P}_{p} \qquad ( 26 )$$

Suppose we have a CCMV virus with a break/deformation pressure of P_{b-CCMV}=477,707 N/m², we can then use (17) to calculate the pulse pressure needed ‘Pₚ’ by setting 100Pₚ=477,707 N/m², which gives P_{b}=4777 N/m\hat{ }2. In water a 200 dB pressure wave in water is equivalent to 10,000 N/m² (about a Large ships broadband emission at 1 meter away), for contrast a Beluga whale about 1 meter away can generate 220 dB which is 100,000 N/m². A 1000 N/m² is equivalent to 180 dB in water, and with a doubling in pressure every 3 dB, then 2000 N/m² is about 183 dB, while 4000 N/m² is about 186 dB. Note that typical active sonar transmission is about 220 dB at 1 m away from the source. Thus a P_{b}=4777 N/m\hat{ }2 is obtainable with the currently available technology.

Therefore, in summary, for modeling, E, kᵥ, F_{threshold}, Δd_{threshold}≈20-30% diameter can be used. Note that exemplary embodiments are not limited to the range Δd_{threshold}≈20-30%, since the target pathogen may have different requirements, so Δd_{threshold} can range Δd_{threshold} between 1-1000%. Additionally, break/deformation pressures, P_{b}, can vary between viruses, bacteria and cancerous cells, and discussion herein is not meant to limit pressures required.

Bacteria Characteristics

The process described is intended to disrupt a pathogen (e.g., virus/bacterium/abnormal/cancerous cells) to a level that renders the pathogen incapable of infecting a host and/or growing effectively. At a minimum, the oscillations discussed need only disrupt the adhesins ability to attach to a host or damage the adhesins themselves, for example if oscillations pass through the host during infection to minimize pathogen infection. To examine the forces needed to disrupt attachment one can look how adhesins proteins on a pathogen interact with the proteins on the host wall, for example Mycobacterium tuberculosis adheres to a host via binding forces that range from 10 pN to 160 pN, with two mode peaks at 50±23 pN and 117±18 pN for each protein binding. The total number of protein bindings will give the total force of adhesion (Gaboriaud F et al., 2005).

The non-limiting example discussed herein examines bacteria. Bacteria, in general, are free-living organisms that typically exist independently from a host, and use internal replication processes. They approach the theoretical size limits for free-living organisms 0.2 μm to several hundred microns in diameter (Morris-Jensen-2008). Each species has a characteristics size and shape, with shape changes common during various phases of development. The current study uses Escherichia coli (E. coli). E. coli, a gram negative bacteria, is typically composed of 70% water and 30% proteins, nucleic acids, ions, and other organic molecules (Goodsell 2009). Gram-negative cells have both an inner (cytoplasmic) membrane and an outer membrane while gram-positive bacteria lack an outer cell membrane but have an overall much thicker cell wall (Morris-Jensen-2008). Membranes tend to relax to a minimum energy configuration which is of simple ellipsoidal shapes, thus variations from such a shape requires mechanical energy. Between the inner and out-membrane is a peptidoglycan layer which is cross-linked and retains its shape in the absence of the rest of the cell, suggesting it is this layer that is responsible for the shape of a bacteria (Morris-Jensen-2008).

A multi-layered cell wall surrounds the cell of an E. coli containing many smaller features such as fimbria (smaller hair like features) and flagella (longer strands for propulsion). The fimbria has sticky ends that attach to human cells and resist attack by the immune system (Goodsell 2009, pg. 58). Damage to fimbria arguably would affect the ability of E. coli to attach to a host and possibly affect its reproduction. When treated with penicillin, bacterial cells lose their shape and ultimately explode under osmotic pressure (Goodsell 2009, pg. 58).

The E. coli flagella is attach to a double-layered cell wall surrounding free floating nucleoid and provides propulsion for the E. coli through liquid environments (Goodsell 2009, pg. 55). The torque generated by an E. coli flagella was measured to be about 700 pN*nm (Darnton N. et al., 2007). Note that in at least one embodiment, the frequency, amplitude, waveform and treatment period are chosen to damage the flagella, reducing the mobility of the E. coli.

For the proof of concept study the strain of E. coli used is DH5-Alpha. DH5-Alpha reproduces by successive binary fission with a generation time of about 30 minutes, with optimum growth occurring at 37° C. (Singh, Om V. et al., 2010). Although the width of E-coli remains stable during growth (1.26 μm±0.16 μm), length changes during growth. For example, stationary (i.e. in a starved no growth condition) E. coli strain BW25113, has an average length of 1.6 μm±0.4 μm, a width of 1.26 μm±0.16 μm, and a volume of 1.5 μm³±1.2 μm³. While the same strain of E. coli, in a growth medium of LB has an average length of 3.9 μm±0.9 μm, a width of 1.26 μm±0.16 μm, and a volume of 4.4 μm³±1.1 μm³ (Volkmer et al., 2011). The reason for the average length differential between E. coli length in a stationary state and in a growth state (in LB) is that E. coli grows by elongation. This is also evident in the standard deviations of the average length which is larger for the E. coli during growth. In at least one exemplary embodiment the target frequency is chosen to be one where the E. coli will grow into the target frequency (i.e. the resonant frequency of the E. coli changes as the E. coli grows and when it matches the target frequency disruption occurs) within a replication period (e.g., 30 minutes).

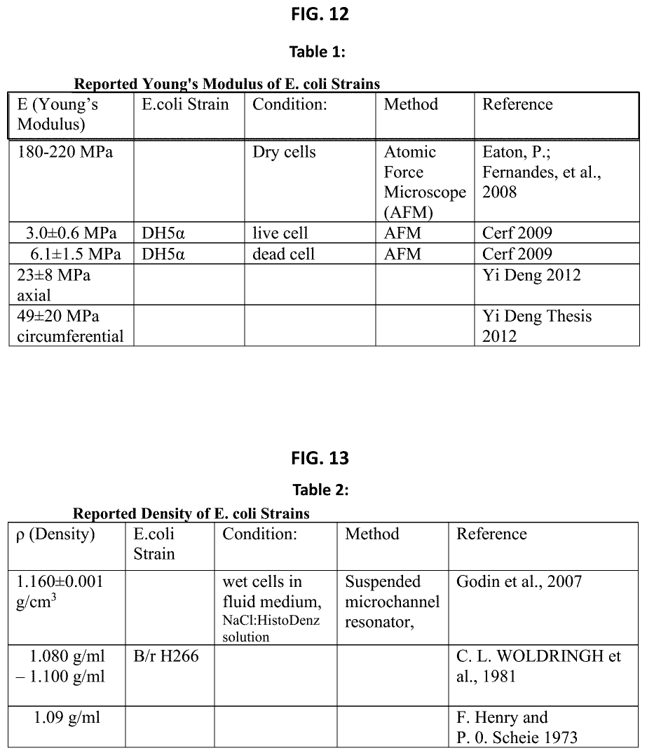

Acoustic properties of bacteria depend upon the composition of the bacteria. As mentioned above when discussing viral characteristics, acoustic properties of pathogens (e.g., viruses, bacteria, cancerous cells, and nonnormal cells) are literature sparse. In material physics a material's resistance to stress and strain in a single direction can be expressed in terms of a Young's modulus (E). The definition of Young's modulus is:

$$E = \frac{stress}{strain} = \frac{F \text{/} A}{Δ L \text{/} L} = \frac{PL}{Δ L} \qquad ( 27 )$$

Where ‘P’ is the pressure acting over area CA in equation 27. The pressure inside (turgor pressure) E. coli has been reported as 29±3 kPa (Yi Deng, 2012). The Young's modulus for E. coli is not well known and the reported value varies from 2 MPa to 220 MPa. FIG. 12 shows Table 1 that summarizes the various reported values for various strains of E. coli. The Young's modulus, E, can be used in a simple spring type model, however, it has been reported that the value of Young's modulus (i.e., elasticity of the cell wall) can vary dependent upon the position along the wall. For example the elasticity of the S. cerevisiae bacteria wall varies significantly across the cell wall from 6 MPa on the bud of the cell wall to 0.6 MPa on the surrounding cell surface (Touhami A, et al., 2003) and (Yves F 2006).

Not only do viruses and bacteria appear to have different mechanically targetable properties but other types of cells do also. For example, it has been reported that various types of cells such as cancerous cells can be distinguished from normal cells based upon their Young's Modulus, for example E=1.97±0.70 kPa for benign mesothelial cells, and 0.53±0.10 kPa for tumor cells (Cross, S. et al., 2007). Hence one can use the current technique by using ultrasonic transmitters on healthy and tumorous tissue. Additionally, it has been reported that when E. coli is infected (predated) with the bacteria virus, Bdellovibrio bacteriovorus, the material properties related to an equivalent spring constant resistant of the cell wall changes from 0.23 N/m, for non-predated to 0.064 N/m predated. Suggesting that even infected bacteria can be targeted separately from health bacteria. Even dead E. coli (6.1±1.5 MPa) have significantly different Young's modulus (E) than living E. coli (3.0±0.6 MPa) of the same strain (Cerf 2009). Basically if there is a resonance frequency difference, one can target a particular pathogen (note that any abnormal cell or foreign body is included when we refer to pathogen, as well as virus and bacteria)

Another property of bacteria is its density, p. FIG. 13 shows Table 2 which lists the various density measurements of E. coli.

The division of E. coli relies on elongation. As discussed above the peptodoglycan layer (P-layer) maintains the shape of a bacteria, thus if growth requires elongation then cutting a circumferential part of the P-layer and filling it in with additional material is needed to produce a rod (Morris-Jensen-2008). If equal materials were not added around the circumferential cut during growth, then the cell would acquire a crescent shape. In at least one exemplary embodiment a target frequency is chosen associated with the condition just prior to replication splitting so as to expose nuclear material to the environment. This frequency can be maintained for the binary fission cycle period so that as the bacteria grows, its resonance frequency or integer fraction, matces the treatment frequency.

### Acoustic Emissions of Bacteria

Several researchers have noticed that bacteria emit sounds at specific frequencies when growing. In 1998 Matsuhashi et al. detected acoustic emissions from Bacillus subtili, with sharp peak formations at 16, 25 and 48 kHz (Matsuhashi M. et al., 1998) Bacillus subtili cells are typically rod-shaped, and are about 4-10 micrometers (μm) long and 0.25-10 μm in diameter, with a cell volume of about 4.6 fL (μm³) at stationary phase (Yu, Allen Chi-Shing et al., 2013). Several studies have detected that microbial growth and metabolism are affected by audible acoustical signals, where in some cases growth is enhanced (Shah et al., 2016). In another study, sound treatments during growth at 1 kHz, 5 kHz and 15 kHz increased the growth of E. coli (cell numbers/ml) to 7%, 34% and 30.5% respectively compared to the average number of viable cells found on the control sample which was 3.70×10⁸ cells per ml. (Ying et al., 2009).

At least one study found that various strains of E. coli generate Acoustic Emissions (AE) when growing (Cox 2014). AE's are stress waves generated by a surge of energy released within an area of a material, due to internal structural changes (Miinshiou et al. 1998). Each E. coli in Cox 2014 was found to generate a unique AE which were measured during growth over a 5-hour period. Another study found that peak frequencies of AE's shifted throughout all phases of growth of two strains (15q and 15cc) of E. coli (Hicks, C. L. et al., 2007). In addition to E. coli other bacteria were found to emit AE's. For example, Lactococcus lactis, an ovoid bacterium of about 0.5 to 1.5 μm, showed a shift in AE peak frequency when inoculated with bacteriophage c2 (a virus that infects bacteria) up to 150 min after inoculation (Cox 2014, pg. 21). It is thought that the AE's in many cases are due to cell wall vibration and not cell resonance.

The resonant theory developed herein indicates that integer fractions of a natural frequency can oscillate and emit depending upon internal damping. The examples discussed in deriving the resonant theory involve external driving forces however internal driving forces can also drive oscillations. For example, in the bacterial growth stage, if internal oscillations are close to the integer fraction of the natural frequency the oscillations will survive, depending upon internal damping. Thus even if multiple integer fraction peaks of the natural frequency can exist, only the AEs dealing with processes close to specific integer fractions will emit for any period of time. A shift in frequencies would be consistent with the resonance theory developed above if material properties change, for example mass or structural integrity (e.g., p, E). Arguably the production of bacteriophages (viruses) in the bacteria changes the density and internal structure (e.g., E) of the host bacteria of Lactococcus lactis, which in accordance to the developed resonance theory above, would change the resonance frequency of the structure and thus the acoustical emission peak frequencies.

Although AE studies are limited, evidence exists that AE's generated by bacteria and bacteriophages are distinct from each other (Cox 2014, pg. 22). If one equates persistent peaked frequency AE's with resonant frequencies of the emitting system, the observed unique AE's support a unique resonance from various types of microbes (e.g., bacteria, viruses (bacteriophage)) as predicted by the resonance theory discussed above.

Cox 2014 studied AE from three strains of E. coli, a parent strain 5024 (coli genetic stock culture number, CGSC #), a mutant strain 8237, and a random/unrelated strain 8279 (Cox 2014, pg. 22). Peak AE frequencies and absolute energy frequencies (ABSE) were detected for the various strains. Peak AE frequencies of about 15.398 kHz, 15.760 kHz, 18.945 kHz, 22.25 kHz, 23.21 kHz, and 28.10 kHz were detected (Cox 2014, pages 61, 64, 65, and 126).

### Models:

Two pulse conditions are examined. Pulse condition 1, where the pulse width (A) is greater than the particle diameter (D), and pulse condition 2 where A is larger than D. For each condition an oscillation model is described and used to acquire target disruption frequencies. For E. coli, even though pulse condition 2 holds, since E. coli replicates in only one dimension, the model of pulse condition 1 is used instead.

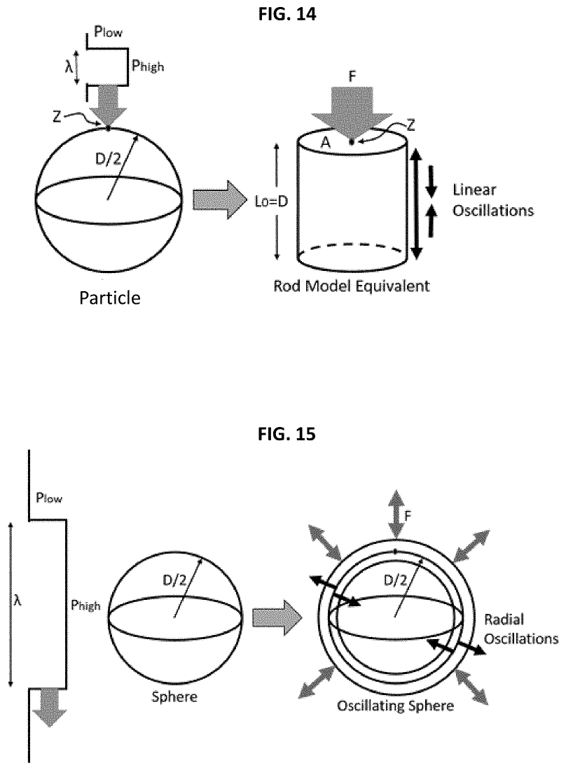

### Pulse Condition 1 (FIG. 14): (Pulse Width λ<Particle Dimension D)

An acoustic wave (e.g., sinusoidal) or pulse train (e.g., square waves) have characteristic single cycle dimension (λ) related to the width of the relative high pressure wave portion. If λ is less than the diameter of the particle/pathogen, then the acoustic driving force can be thought of as a one-dimensional driving force F. FIG. 14 illustrates a one-dimensional linear oscillation model when pulse condition 1 applies (λ<D). To determine the characteristic area A, a simplified model of a sphere is used and the volume of the sphere can be matched to the volume of the one-dimensional rod model. For an actual pathogen, the volume of the pathogen is matched to the rod model volume (eqn. 2.1.1).

$$\frac{4}{3} {π ( \frac{D}{2} )}^{3} = {AL}_{0} = AD \qquad ( 2.1 .1 )$$

Solving for the area A, one obtains:

$$A = \frac{π {D}^{2}}{6} \qquad ( 2.1 .2 )$$

The linear elastic property (Young's Modulus, E) of a rod is defined as:

$$E = \frac{( \frac{F}{A} )}{( \frac{Δ L}{{L}_{eff}} )} \qquad ( 2.1 .3 )$$

The linear oscillation can be expressed as:

$$F = k Δ L = ( \frac{EA}{{L}_{eff}} ) Δ L \qquad ( 2.1 .4 )$$

The natural frequency, f₀, can be expressed as:

$${f}_{0} = \frac{{ω}_{0}}{2 π} = \frac{1}{2 π} \sqrt{\frac{k}{m}} = \frac{1}{2 π} \sqrt{\frac{EA}{{mL}_{eff}}} = \frac{1}{2 π} \sqrt{\frac{E}{m} \frac{\frac{π {D}^{2}}{6}}{D}} = \frac{1}{2 π} \sqrt{\frac{π ED}{6 m}} \qquad ( 2.1 .5 )$$

The integer fraction (1/n) of the natural frequency can be expressed as:

$${f}_{n} = \frac{{ω}_{0}}{2 π} ( \frac{1}{n} ) = ( \frac{1}{n} ) \frac{1}{2 π} \sqrt{\frac{k}{m}} = ( \frac{1}{n} ) \frac{1}{2 π} \sqrt{\frac{π ED}{6 m}} = ( \frac{1}{n} ) \frac{1}{2 π} \sqrt{\frac{π E ( \frac{1}{3} ) \frac{D}{2}}{\frac{4}{3} {π ( \frac{D}{2} )}^{3} ρ}} = ( \frac{1}{n} ) \frac{1}{2 π D} \sqrt{\frac{E}{ρ}} \qquad ( 2.1 .6 )$$

Summarized as:

$${f}_{n} = ( \frac{1}{n} ) \frac{1}{2 π D} \sqrt{\frac{E}{ρ}} Pulse Condition 1 ( λ < D ) \qquad ( 2.1 .7 )$$

Two materials, polyethylene and polystyrene, before E coli testing, are used to test the models and concepts discussed herein. Young's modulus (E) of polyethylene HDPE is about 0.8 GPa, the bulk modulus (B) of polyethylene is about 0.14 GPa, and the density of about 0.96 g/cc. The Young's modulus (E) of polystyrene is between about 3.0-3.5 GPa, bulk modulus (B) of polystyrene is about 4 GPa and density of about 1.0507±0.0004 g/cm³. Equation 2.1.7 for polyethylene and polystyrene can be expressed as:

$$\begin{matrix}{f}_{n} = ( \frac{1}{n} ) \frac{1}{2 π D} \sqrt{\frac{E}{ρ}} = ( \frac{1}{n} ) ( \frac{1}{2 π D} ) \sqrt{\frac{0.8 \times {10}^{9} Kg \text{/} {ms}^{2}}{960 Kg \text{/} {m}^{3}}} = 912.87 m \text{/} s ( \frac{1}{n} ) ( \frac{1}{2 π D} ) Polyethylene , Pulse Condition 1 ( λ < D ) & ( 2.1 .7 A ) \\ {f}_{n} = ( \frac{1}{n} ) \frac{1}{2 π D} \sqrt{\frac{E}{ρ}} = ( \frac{1}{n} ) ( \frac{1}{2 π D} ) \sqrt{\frac{3.25 \times {10}^{9} Kg \text{/} {ms}^{2}}{1050.7 Kg \text{/} {m}^{3}}} = 1758.74 m \text{/} s ( \frac{1}{n} ) ( \frac{1}{2 π D} ) Polystyrene , Pulse Condition 1 ( λ < D ) & ( 2.1 .7 B )\end{matrix}$$

The ‘n’ fractional resonant frequency of a 655-micron polyethylene sphere using the acoustic wave model, as discussed above is:

$${f}_{nr} = \frac{{c}_{1}}{n 2 d} = \frac{2430}{n \cdot 2 \cdot 655 \times {10}^{- 6}} = \frac{1.8550 MHz}{n} \qquad ( 28 )$$

The equivalent elastic rod model for pulse condition 1 is:

$${f}_{n} = 912.87 ( \frac{1}{n} ) ( \frac{1}{2 π \times 655 \times {10}^{- 6}} ) = \frac{221813.39 Hz}{n} \qquad ( 2.1 .7 A1 )$$

for Polystyrene, Pulse Condition 1 (λ<D), for 655-micron diameter sphere.
The value predicted by equation (2.1.7A1) is a factor 0.1196 of the predicted value of equation (28).

The acoustic model for resonance of polystyrene 1.95 mm diameter sphere, is:

$${f}_{nr} = \frac{{c}_{1}}{n 2 d} = \frac{2407}{n \cdot 2 \cdot ( 1.95 \times {10}^{- 3} )} = \frac{617179.4872 Hz}{n} \qquad ( 29 )$$

The equivalent elastic rod model for pulse condition 1 is:

$${f}_{n} = 1758.74 ( \frac{1}{n} ) ( \frac{1}{2 π \times 1.95 \times {10}^{- 3}} ) = \frac{143544.70 Hz}{n} \qquad ( 2.1 .7 B1 )$$

for Polystyrene, Pulse Condition 1 (λ<D), for 1.95 mm diameter sphere.
The value predicted by equation (2.1.761) is a factor 0.2326 of the predicted value of equation (29). Experiments with particles are tested at both acoustic and elastic predictions, while E. coli experiment use elastic models.

### Pulse Condition 2 (FIG. 15): (Pulse Width λ>Particle Dimension D)

For pulse condition 2, λ>D, the particle (e.g., sphere) is radially oscillated. The radial oscillation elastic property is expressed by a bulk modulus, B, which is defined as:

$$B = \frac{Δ P}{Δ V \text{/} V} \qquad ( 2.1 .8 )$$

Where ΔP is pressure and V is volume. Relating pressure to F/A, and spherical volume for V one obtains:

$$B = \frac{F \text{/} A}{Δ V \text{/} V} = \frac{F \text{/} A}{Δ V \text{/} V} = \frac{F \frac{V}{A}}{Δ V} = F ( \frac{1}{Δ V} ) \frac{\frac{4}{3} π {r}^{3}}{4 π {r}^{2}} = F ( \frac{1}{Δ V} ) \frac{r}{3} \qquad ( 2.1 .9 )$$

Equation 2.1.8 can be rewritten solving for the force F, as

$$F = \frac{3 B}{r} Δ V \qquad ( 2.1 .10 )$$

To obtain the radial oscillation properties one needs to express equation 2.1.9 in the form of F=kΔr. The change in volume ΔV can be expressed in terms of Δr as:

$$Δ V = {V}_{ave} - {V}_{min} = \frac{4}{3} π ( {r}^{3} - {( r - Δ r )}^{3} ) = \frac{4}{3} π r \{ 3 r Δ r - 3 Δ {r}^{2} + \frac{Δ {r}^{3}}{r} \} \qquad ( 2.1 .11 )$$

If we assume that the linear deformation is at most 20%, then using Δr=0.2r, the terms of equation 2.1.11 can be expressed as:

$$\begin{matrix}3 r Δ r = 0.60 {r}^{2} & ( 2.1 .12 ) \\ 3 Δ {r}^{2} = 0.12 {r}^{2} & ( 2.1 .13 ) \\ \frac{Δ {r}^{3}}{r} = 0.008 {r}^{2} & ( 2.1 .14 )\end{matrix}$$

If we keep just the first term we have:

ΔV=Vₐᵥₑ−Vₘᵢₙ=≈4/3π3r²Δr (2.1.15)

Using eqn. 2.2.15 in 2.1.9 we obtain:

$$F \approx \frac{3 B}{r} ( \frac{4}{3} π3 {r}^{2} ) Δ r = ( 12 π Br ) Δ r \qquad ( 2.1 .16 )$$

The natural frequency can then be expressed as:

$${f}_{0} = \frac{{ω}_{0}}{2 π} = \frac{1}{2 π} \sqrt{\frac{k}{m}} \approx \frac{1}{2 π} \sqrt{\frac{12 π Br}{m}} = \sqrt{\frac{12 π Br}{4 {π}^{2} m}} = \sqrt{\frac{3 Br}{π ( ρ \frac{4}{3} π {r}^{3} )}} = \frac{1}{π} ( \frac{1}{r} ) \frac{3}{2} \sqrt{\frac{B}{ρ}} \qquad ( 2.1 .17 )$$

And the fraction integer can be expressed as:

$$\begin{matrix} {f}_{n} = \frac{{ω}_{0}}{2 π} ( \frac{1}{n} ) \approx ( \frac{1}{n} ) \frac{3}{2 π r} \sqrt{\frac{B}{ρ}} Pulse Condition 2 ( λ > D ) & ( 2.1 .18 ) \\ {f}_{n} \approx ( \frac{1}{n} ) \frac{3}{2 π r} \sqrt{\frac{B}{ρ}} = ( \frac{1}{n} ) \frac{3}{2 π r} \sqrt{\frac{\frac{0.14 \times {10}^{9} Kg}{{ms}^{2}}}{960 \frac{Kg}{{m}^{3}}}} = 381.88 m \text{/} s ( \frac{1}{n} ) ( \frac{6}{2 π D} ) = 2291.29 m \text{/} s ( \frac{1}{n} ) ( \frac{1}{2 π D} ) for Polyethylene , Pulse Condition 2 ( λ > D ) . & ( 2.1 .18 A ) \\ {f}_{n} \approx ( \frac{1}{n} ) \frac{3}{2 π r} \sqrt{\frac{B}{ρ}} = ( \frac{1}{n} ) \frac{6}{2 π r} \sqrt{\frac{\frac{4.00 \times {10}^{9} Kg}{{ms}^{2}}}{1050.7 \frac{Kg}{{m}^{3}}}} = 11706.90 m \text{/} s ( \frac{1}{n} ) ( \frac{1}{2 π D} ) for Polystyrene , Pulse Condition 2 ( λ > D ) & ( 2.1 .18 B )\end{matrix}$$

E. coli Models

Now we examine the use of acoustic/pressure waves on E. coli at specific model determined frequencies. The pulse widths/wavelengths of the acoustic source signals are limited by the sound generating equipment, for example a function generator's time width of generated pulses, and a transducer's capacity to convert the function generator signal into an equivalent sound pulse/wave. The equipment available for this study could generate pulse widths of the order of about 10 microns, much larger than a typical viral dimension and larger than most bacteria. Since the pulse width (λ) was larger than the pathogen characteristic size (D), only pulse condition 2 (λ>D) was used. E. coli was chosen as the pathogen because of its common usage in biological studies. The DH5α E. coli strain was used throughout this study because of availability and some of the properties (e.g., Young's modulus) have been reported in references.

At least one exemplary embodiment applies the acoustic techniques, to affect the growth of E. coli. The growth nature of E. coli presents challenges. The resonance techniques depend on pathogen dimension, however the length of E. coli changes when it replicates thus preventing the application of a single frequency to eradicate the entire sample of E. coli if the exposure time is less than a full cycle of growth, about 25 minutes for E. coli. The biological method of analyzing E. coli bacterial growth uses optical density (OD) of the dispersion of 600 nm wavelength light through a bacterial sample. Bacterial numbers have a linear relationship to optical density. As the bacteria grows the optical density value increases in a power law fashion. Understanding how eradication of a portion of the initial bacteria is reflected in OD plots is modelled below, then a resonance model for E. coli is developed.

### Bacterial Growth Curves and Growth Model

Mathematical models have traditionally been used to facilitate the interpretation of bacterial growth curves in order to more accurately understand and identify variations in bacterial growth rates. This study develops a growth model that takes into account size variation during binary fission and incorporates specific size eradication in growth models. The binary fission bacterial growth model describes a multi-bin growth mode, utilizing Escherichia coli (DH5α) as an experimental model, where each bin is associated with a size range during E. coli growth cycle. Comparisons between the theoretical model and experimental observations demonstrates that bacterial growth curves and the ratio of sample growth curve to control growth curves can be used to determine and normalize initial variations in bacterial levels among test samples, as well as identify final nutrient levels, and the percentage of bacteria compared to control levels.

Analytical models have increasingly been applied to bacterial growth data in an effort to more accurately interpret bacterial growth characteristics during sample testing (Rickett et al., 2015). Analyzing bacterial growth typically relies on either direct methods, relying on counting, or through indirect methods such as measuring turbidity through measurement of the optical density (OD) of bacterial suspensions. High throughput sample testing often relies on multi-well plate formats (e.g., 96-Well Plate). Various wells have bacterial samples, standard controls, and blanks. Due to variations in bacterial sample starting numbers, comparing the growth curves of treated samples with control samples can be difficult if initial bacterial levels are not identical. Even the most precise sample dispensation techniques can result in variations of initial bacterial levels and/or nutrient broth levels, which can be further compounded with further biological and environmental variability. Comparing the growth curves of treated samples with control samples can be difficult if initial bacterial levels are not identical. Similarly, when nutrient broth is injected into post treatment sample wells the final growth levels will be a function of the available nutrient levels. The binary fission model developed here examines simulated growth curves and growth curve ratios for various initial bacterial levels and nutrient broth levels, developing tools for correcting variations in initial bacterial levels and nutrient broth levels.

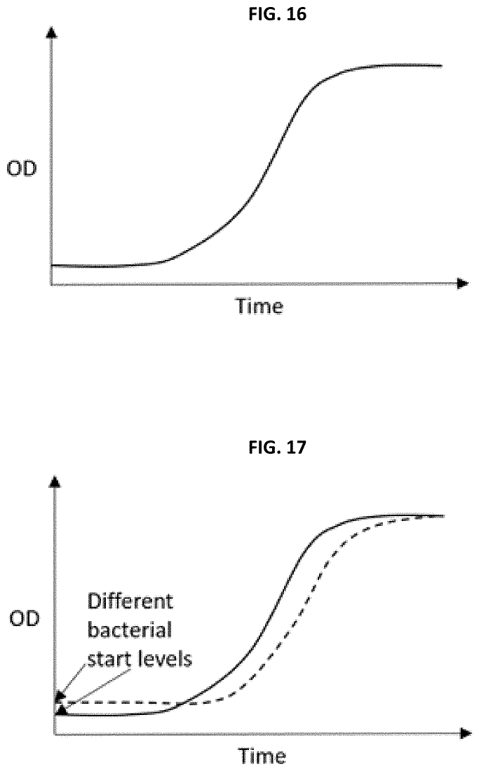

Growth curves typically undergo a lag, exponential, and stationary phase. FIG. 16 illustrates a classical growth model developed by Baranyi and Roberts (Baranyi et al., 1993, Baranyi et al., 1994, Ricket et al., 2015) illustrating E. coli propagates from binary fission. The size of the E. coli changes during binary fission. At a particular size the E. coli replicates. Providing a range of sizes from initial replication to the next fission. An individual E. coli bacterium grows essentially in one dimension (longitudinal) prior to fission, while remaining essentially unchanged in the axial (width) dimension. Hence at any given time a range of bacterium size occurs from a minimum size just after fission to a maximum size just prior to fission. The size then is directly associated with the time till next fission.

The width of E-coli remains stable during growth and is about 1.26 μm±0.16 μm (Volkmer et al., 2011). For example, stationary (i.e., in a starved no growth condition) E. coli strain BW25113, has an average length of 1.6 μm±0.4 μm, a width of 1.26 μm±0.16 μm, and a volume of 1.5 μm³±1.2 μm³. While the same strain of E. coli, in a growth medium of LB has an average length of 3.9 μm±0.9 μm, a width of 1.26 μm±0.16 μm, and a volume of 4.4 μm³±1.1 μm³ (Volkmer et al., 2011). The reason for the average length differential between E. coli length in a stationary state and in a growth state (in LB) is that E. coli grows by elongation. This is also evident in the standard deviations of the average length which is larger for the E. coli during growth.

Many growth models have sought to simulate various aspects of bacterial growth curves. Rickett et. Al. 2015 seeks to use Bayesian statistics to detect differences between bacterial growth rates, using a 4 parameter Baranyi and Roberts model (Baranyi et al., 1993, Baranyi et al., 1994, Rickett et al., 2015). Huang seeks to model, the lag, exponential and transition phases, using exponential and logarithm functions and compares the developed model to the Baranyi and Roberts Model (Huang, 2008, Huang 2010). In each model the growth is not treated as separately binned populations, where each bin includes a population of size range of E. coli during growth. In the model presented here, the population in each bin gradually increases until it then populates the next bin, until reaching the maximum size bin. After the maximum size bin each E. coli undergoes binary fission and the lowest bin is increased in population.

Since current growth models do not examine size specific eradication, nor do they provide a realizable method for identifying initial differences in bacterial levels between samples and controls, a size specific model that provides a method of determining percentage of bacteria eradicated during treatment if the treatment is specific to a stage of binary fission, and provides a means for identifying and correcting curves for initial bacterial differences.

Bacterial growth, assuming no deaths, can be expressed as:

N(t)=N₀2^{(t/t}^{_{dbl}}⁾ (2.2.1)

Where N(t) is the bacteria number at some time t, from the start bacterial number N₀, where t_{dbl} is the bacterial time of duplication. In the current study untreated bacteria can be used as control (C), whose growth can be expressed as:

N_{c}(t)=N_{0c}2^{(t/t}^{_{dbl}}⁾ (2.2.2)

The growth of the treated bacteria (T) can be expressed as:

N_{T}(t)=N_{0T}2^{(t/t}^{_{dbl}}⁾ (2.2.3)

The Optical Density (OD) is directly related to the bacterial number N(t), where OD can be plotted versus time to examine bacterial growth (FIG. 16).

Another method of examining the effect of variations in initial conditions such as initial bacterial amounts and variations in food is to take the ratio of the numbers of treated bacteria (Eqn. 2.2.3) to control bacterial amounts (Eqn. 2.2.2). The ratio of time dependent growth, if times are not simultaneously measured, for example for a treated sample measured at time t1 and control measured at time t, can be expressed as:

$$\frac{{N}_{T} ( {t}_{1} )}{{N}_{c} ( t )} = \frac{{N}_{0 T} {2}^{( {t}_{1} \text{/} {t}_{dbl} )}}{{N}_{0 c} {2}^{( t \text{/} {t}_{dbl} )}} \qquad ( 2.2 .4 )$$

If the number of bacteria are measured near simultaneously, t1≈t, for example in an Optical Density 600 nm measuring device, the ratio can be expressed, independent of time, as:

$$\frac{{N}_{T} ( t )}{{N}_{c} ( t )} = \frac{{N}_{0 T}}{{N}_{0 c}} \qquad ( 2.2 .5 )$$

Multiplying the result of equation (2.2.5) by 100 gives the % of the initial amount of treated bacteria compared to the control bacteria, taking into account experimental errors in attempting to set equal initial bacterial levels. FIG. 17 illustrates an OD curve of a treated sample (dashed line) versus control (solid line), where the treated sample has an initial amount of bacteria that is larger than the control but, for this example, the same amount of food. Note that since the sample has been treated the onset of growth occurs later since some of the initial pathogen has been destroyed by the treatment.

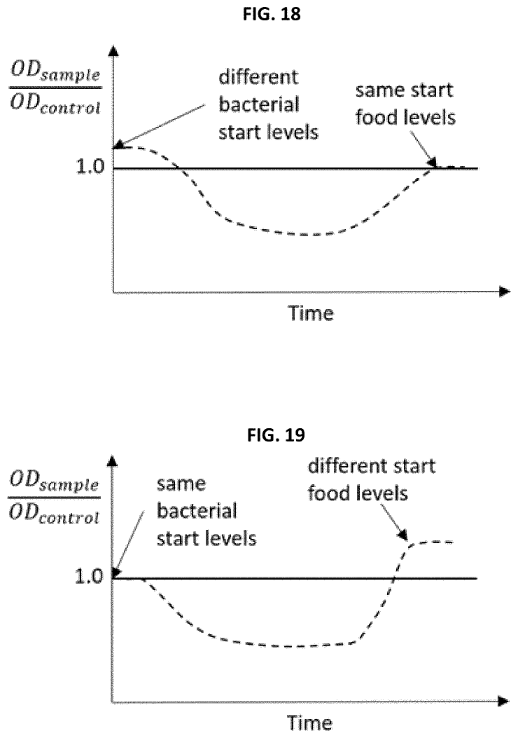

FIG. 18 illustrates the ratio (eqn. 2.2.5) of the OD curves illustrated in FIG. 17. the solid line at a value of 1.0 represents the ratio of control to itself. At lower times, the relative initial bacterial amounts can be viewed.

Bacterial growth is eventually limited by available food. Thus even if a portion of the initial bacteria is destroyed, it will eventually grow to near the same amount as the control if the food amount is the same. The initial errors in the attempt to set equal food levels can be examined by viewing the ratio at the times where the control bacterial growth levels plateau. To examine the ratio at larger times, food contribution must be taken into account. If we include the limitation that growth is limited by available food, then Food (F) usage can be expressed, where F0 is the total amount of bacteria that can replicate using the available food, as:

F(t)=F₀−N₀2^{(t/t}^{_{dbl}}⁾ (2.2.6)

If we set F(t) to 0.0 then we can solve for the time, tₗ, it takes for the food to run out.

$$\begin{matrix}0.0 = {F}_{0} - {N}_{2} {2}^{( t \text{/} {t}_{dbl} )} & ( 2.2 .7 ) \\ \frac{{F}_{0}}{{N}_{0}} = {2}^{( {t}_{l} \text{/} {t}_{dbl} )} & ( 2.2 .8 )\end{matrix}$$

To solve for tₗ, one can take the log base 2 of the left side of equation 2.2.8:

$$\begin{matrix}{log}_{2} ( \frac{{F}_{0}}{{N}_{0}} ) = \frac{{t}_{l}}{{t}_{dbl}} & ( 2.2 .9 ) \\ {t}_{l} = {t}_{dbl} {log}_{2} ( \frac{{F}_{0}}{{N}_{0}} ) & ( 2.2 .10 )\end{matrix}$$

Substituting equation 2.2.10 into equation 2.2.1, we get:

$$\begin{matrix}N ( {t}_{l} ) = {N}_{0} {2}^{( {t}_{l} \text{/} {t}_{dbl} )} = {N}_{0} {2}^{( {t}_{dbl} {log}_{2} ( \frac{{F}_{0}}{{N}_{0}} ) \text{/} {t}_{dbl} )} & ( 2.2 .11 ) \\ N ( {t}_{l} ) = {N}_{0} {2}^{{log}_{2} ( \frac{{F}_{0}}{{N}_{0}} )} = {F}_{0} & ( 2.2 .12 )\end{matrix}$$

If various control wells have different levels of initial food, for example sample initial food F₀ₛ and control initial food F_{0c}, one can take the ratio of samples with respect to the control to get the ratio of the initial food amounts at time t≥t₁. This can be expressed as:

$$\frac{{N}_{S} ( {t}_{ls} )}{{N}_{c} ( {t}_{lc} )} = \frac{{F}_{0 S}}{{F}_{0 c}} \qquad ( 2.2 .13 )$$

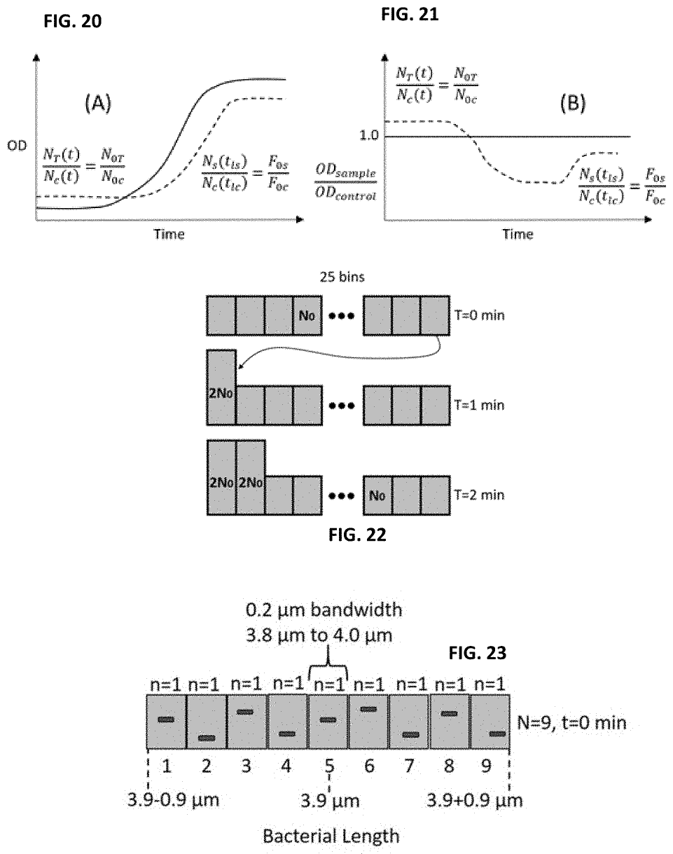

The experimental condition where the initial bacterial amounts match but the initial food (e.g., LB broth) levels were different can be examined using the ratio (eqn. 2.2.13) and is illustrated in FIG. 19 for the condition where the food of the sample is larger than the control. Using the ratio of equation 2.2.13 one can read directly the initial bacterial and food levels with respect to controls. The initial bacterial levels can be adjusted so that direct comparison of the effect of bacterial treatment can be compared to control values, where both start at the same initial value. FIGS. 20 and 21, summarize the various regions of the OD curve and where the ratio can be used to examine initial bacterial levels, and relative food levels. One advantage of using the ratio plots is the ability to read directly from the plots the relative amount with respect to control values. Growth models (described below) can be used to understand how various levels of the initial conditions compared to actual bacterial eradication influences the OD and OD-ratio curves. FIGS. 20 and 21 illustrate plots with sample treatment. The middle plateau in FIG. 21 is directly related to the amount of bacteria eradicated during treatment, once the initial bacterial levels are adjusted. The shape of the curve is dependent on whether bacteria of all sizes (i.e., various sizes during binary fission) are equally treated or only a size range treated, as discussed below.

### Bacterial Growth BIN Model

The acoustic resonance disruption of E. coli is tailored for a particular size per frequency. Since E. coli has various sizes during growth, only a portion of the E. coli will be affected at a particular frequency. To examine the effect of disrupting a portion of the starting bacterial colony, a bin model has been developed, where each bin represents the number population of a specific discrete size. Since E. coli is largest just before replication, the larger discrete sized bins are associated with the bacteria about to replicate, whereas the smaller sizes are associated with bacteria that have nearly a full cycle of growth before replication. On average E. coli takes about 25 minutes to duplicate. So using twenty-five (25) bins, bin 25 represents the number of bacteria that will replicate in 1 minute, while bin 1 represents the number of bacteria that will replicate in 25 minutes. FIG. 22 represents the basic BIN model of the initial bacteria in a sample.

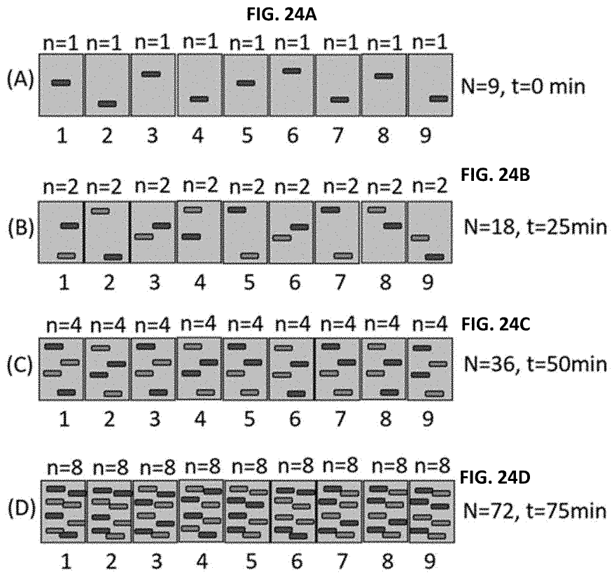

Using the Bin model, we can examine the effect of eradicating bacteria of a particular size while leaving other sizes unaffected. FIG. 23 illustrates an example of a bin model with 9 bins, where each bin is associated with a certain range of length of bacteria. For example, for E. coli the average length during growth is 3.9 μm with a standard deviation of 0.9 μm. If the bin model covers the mean plus or minus one standard deviation, each bin encompasses a 0.2 μm bandwidth. For example, bin 5 encompasses a bacterial length that ranges 3.8 μm to 4.0 μm, with each length associated with a unique resonant frequency. E. coli, reproduces by binary fission, meaning 1 bacteria turns into 2 after a certain period, about 25 minutes for E. coli. FIGS. 24 (A)-(D) illustrate the growth per bin if every 25 minutes the population doubles in each bin.

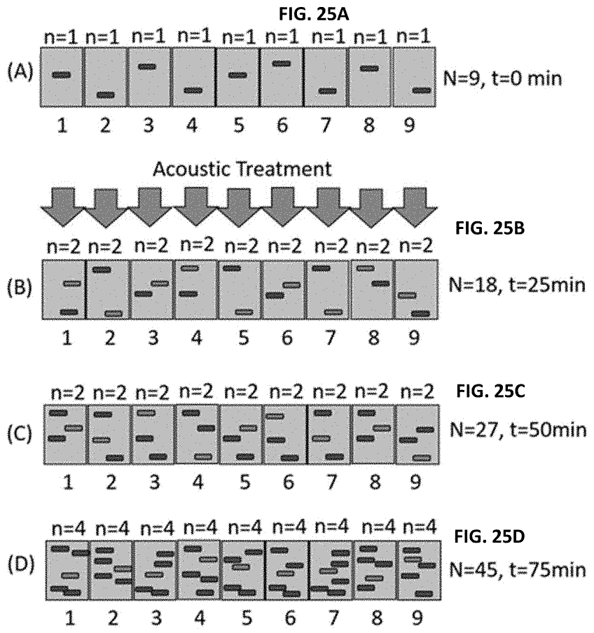

The bacterial colors are illustrated in red and blue to indicate later the effect of targeting (blue) a particular percentage of the bacteria in a bin, starting after the first replication at time t=25 min. In FIGS. 24 A-D no treatment occurs so the bacterial number grows from N=9 (A) to N=18 (B) after 25 minutes, then N=36 (C) at 50 minutes, and N=72 at 75 minutes. In the current example, initially each bin has one bacteria in it (FIG. 25 (A)) and after 25 minutes each bin has two bacteria (FIG. 25 (B)), after which treatment occurs. In the model displayed in FIG. 25 (B) treatment is assumed to affect all bins equally targeting half of the bacteria (for model considerations), the blue bacteria. The blue bacteria no longer can undergo binary fission, even though the Optical Dispersion at 600 nm (for E. coli) still is effect by the sterile blue bacteria. After treatment at time 25 minutes (B) only the red (non targeted bacteria) undergoes binary fusion at times 50 (C) and again at 75 minutes (D). FIGS. 25 (B)-(D) illustrate the condition where each bin is treated equally.

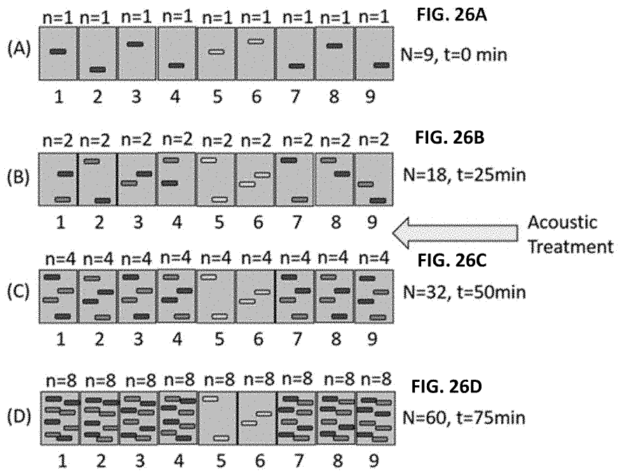

A more realistic model focuses on a limited number of bins associated with a unique resonance frequency. This is illustrated in FIGS. 26 A-D. The red and blue bacteria are similar to that shown in FIGS. 25 A-D, while the yellow bacteria is meant to symbolized the targeted bacteria, which could be a particular size range of bacteria since each bin is associated with a size range. After treatment at time=25 min only the non-treated bins undergo binary fission. Note that the bacterial count is N=45 at 75 min with uniform treatment (FIG. 25 (D)), N=60 at t=75 min with bin focused treatment (FIG. 26 (D)), while an untreated bacterial number of N=75 occurs at 75 min (FIG. 24 (D)). Note that in reality the bacteria from bin 7 in FIG. 26C, would grow into bin 8 in FIG. 26C, thus even more realism can be obtained by modifying the concept illustrated in FIGS. 26 A-D so that each bin is associated with a size range and thus associated with a time to binary fusion of that particular bin.

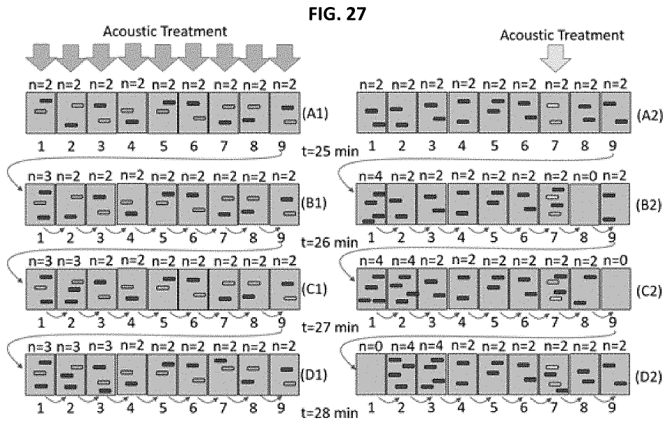

For example, FIGS. 27 A1-D1 illustrate uniform treatment but also bin dependent fission. For example, bin 9 includes the largest size bacteria ready to replicate. Hence 1 minute later at t=26 min only the non-treated bacteria (red) in bin 9 undergoes fission breaking up the largest bacteria in bin 9 to an increased number in the lowest size bin 1 (n=3), while the bacteria in each bin grows and moves into the next bin category. For example, the new smaller bacteria previously from bin 9 now populates bin 1 while bin 2 bacteria, now larger, populates bin 3 and so on. A minute later the non-treated bacteria in bin 9, previously from bin 8, undergoes fission. In the uniform treated version illustrated in FIGS. 27 A1-D1 the bacterial growth is constant in time. In the bin or size specific treatment (FIGS. 27 A2-D2) the bacterial number between time t=27 min and t=28 min does not change since there is a gap in live bacteria due to targeted treatment. Thus in an OD plot one would expect a constant periodic plateaus for a short period of time until the lack of live bacteria gap has passed. The onset of the plateau can provide information as to what size (bin number) the treatment was most effective at. For example, if the treatment eradicated the largest size (bin 9) then the plateau occurs almost immediately, while a smaller size affected bin (e.g., bin 1) would result in a later onset of the plateau.

### Potential Sources of Error in Bacteria Experiments

E. coli experiments were performed in 96-well trays, where initial bacteria levels were pipetted into select wells. There can be differences in initial bacterial levels, due to pipetting differences. Additionally, after treatment, LB growth medium (food levels) are inserted to facilitate the growth of any remaining live bacteria. Errors in the pipetting amounts of the after treatment growth medium inserted into a well can result in differences in final food amounts. Simulations below using the Bin model examines the various experimental errors in initial bacterial and final food level. In summary the ratio of the OD values of sample to control provides a method to examine variation in initial bacterial levels and final food levels.

### Simulated OD and OD-Ratio Curves for Various Eradication Levels: Identical Initial Live Bacterial Levels and Identical Final Food Levels.

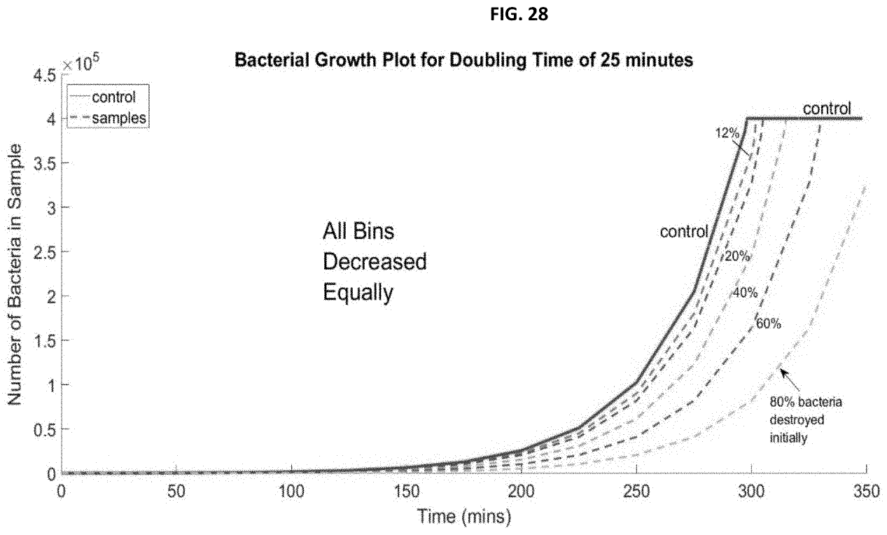

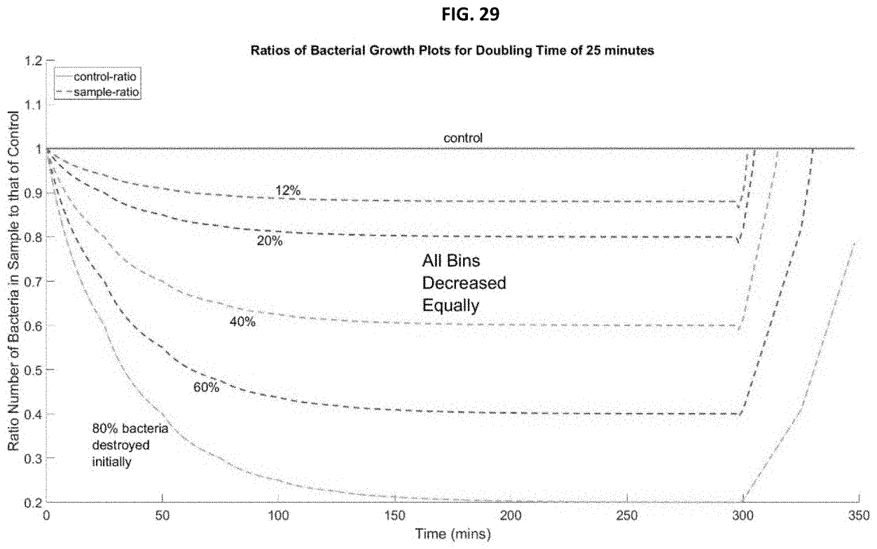

OD curves are often used in peer-reviewed-journal articles to examine bacterial growth over time. FIG. 28 illustrates simulated growth curves for various initial eradication levels (e.g., resulting from acoustical treatment) from the Bin Model assuming any initial bacterial eradication is distributed equally amongst bins. The modelling conditions resulting for FIG. 28 have identical initial live bacterial levels (prior to treatment) and identical final food levels. Note the obvious delay of the onset of logarithmic portion of growth (between about 175 minutes to 275 minutes) as a function of eradication level. Identical initial bacterial and final food levels can more easily be seen in the OD-ratio plot of FIG. 29.

The identical bacterial levels (OD-ratio equal to 1) can be seen by looking at the OD-ratio levels at 0 minutes. During the logarithmic level stages of the OD plot of FIG. 29, the OD-ratio value converges to the relative live bacterial levels that remain after treatment. Finally, the relative final food levels can be examined by looking for the final plateau value after the control values plateau, about 300 minutes for the control and 325 minutes for the 60% bacteria eradicated curve. Errors in initial bacterial amounts and final food levels can also result in onset variations in logarithmic portions of OD curves, these will be examined in the following sub sections.

### Simulated OD and OD-Ratio Curves for Various Eradication Levels: Identical Initial Live Bacterial Levels and Different Final Food Levels.

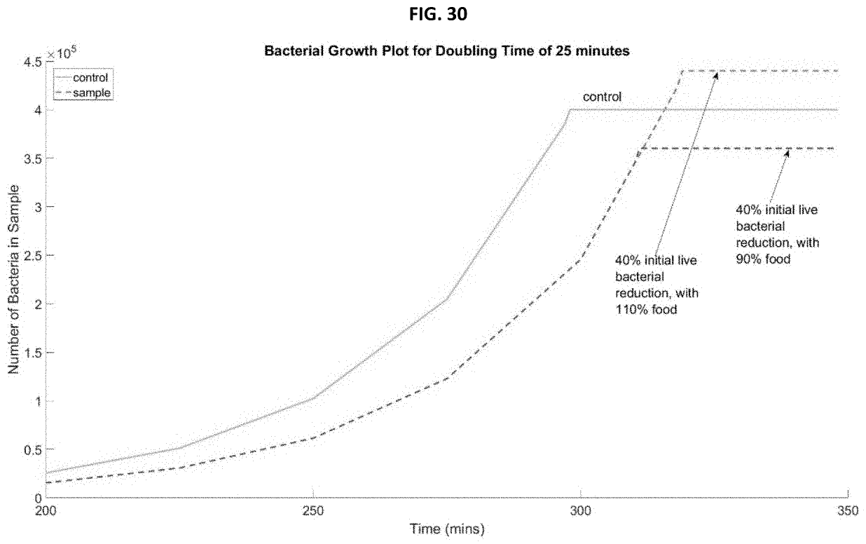

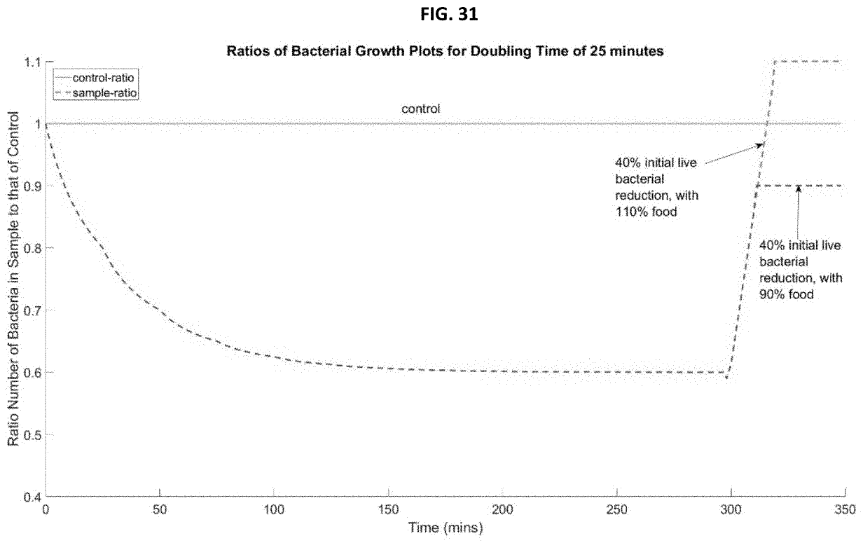

FIG. 30 illustrates simulated bacterial number, which are directly related to OD values, curves for three final food conditions, 110% more in the treated sample than that for the control condition, 100% of the control condition and 90% of the control condition. As can be seen the plateau of the bacterial numbers, which are directly related to OD values, levels off at levels related to the final food available, which limits the final number of bacteria. This can be more easily seen in the ratio of bacterial levels of a sample to the level for the controls as shown in FIG. 31. The ratio curves of FIG. 31 clearly indicate the various relative final food amounts. For example, the three plateaus occur at ratio values of 1.1, 1.0 and 0.9, related to 110%, 100% and 90% of the control food levels. The advantage of the ratio curves is that the relative amounts can be directly obtained.

### Simulated OD and OD-Ratio Curves for Various Eradication Levels: Identical Initial Live Bacterial Levels and Different Final Food Levels.

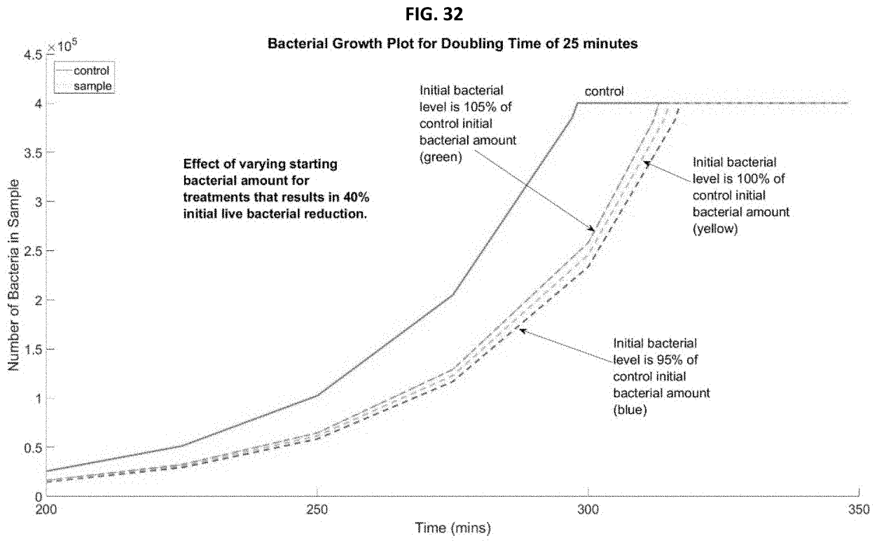

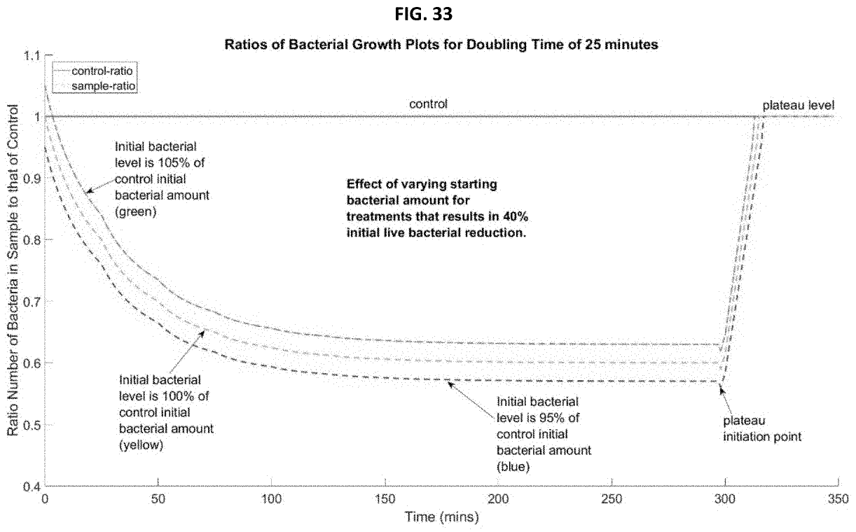

FIG. 28 shows the variation of simulated OD curves as a function of eradicating a portion of the initial bacterial levels by treatment. Experimental errors, such as variations in initial bacterial levels, can also result in OD curve variations in the logarithmic growth regions. FIG. 32 shows simulated OD curves for three initial bacterial level variations of 105%, 100%, and 95% of the control levels for the 40% eradication of initial bacteria condition. Note that a variation in the OD curve has been shifted in FIG. 32, however it is difficult to obtain the amount of variation of the initial bacterial levels from the graph. The actual levels of initial live bacteria are better observed in the simulated ratio plots of FIG. 33, which can be obtained from the 0 min values. Note that even though the zero-time ratio values of FIG. 33 can be used to determine relative initial bacterial levels, the plateau time values, for example after 300 minutes, indicate that the final food levels are identical.

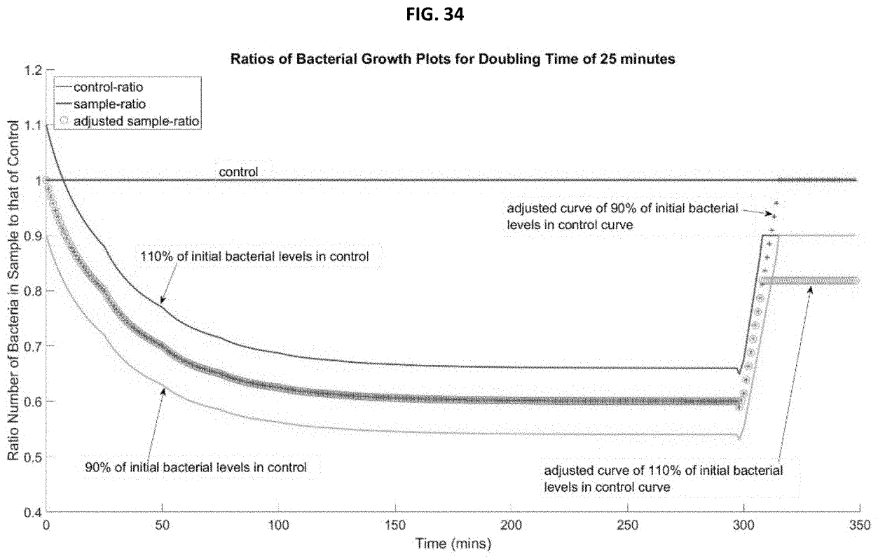

To minimize errors, the ratio OD plots can be used to adjust plots to take into account variation of initial bacterial levels. Only the portion up to the plateau initiation point need be adjusted since the initial bacterial levels will not affect the final bacterial levels which are dependent upon the final food levels. FIG. 34 shows the effect of shifting the simulated OD-ratio curves of FIG. 33. The ratio adjusted values (RA) can be expressed as:

$${RA}_{t = 0} = {( \frac{{OD}_{sample}}{{OD}_{control}} )}_{t = 0} , \qquad ( 2.2 .14 )$$

where the zero-time (t=0 sec) ratio values represent the initial bacterial differences.

Note that the adjusted ratio curves (circles and purple stars) now have accurate converging values of 0.6, however adjusting the curves using the initial ratio values results in inaccurate final plateau bacterial levels. Thus when examining eradication levels compared to the control the ratio curves should be adjusted by using the time=0 sec ratio data, but if the final bacterial growth levels are desired then the curves should not be adjusted.

Simulated OD and OD-Ratio Curves for Various Eradication Levels: Identical Initial Live Bacterial Levels and Final Food Levels, where Only a Specific Range of Bins in the Bin Model are Eradicated.

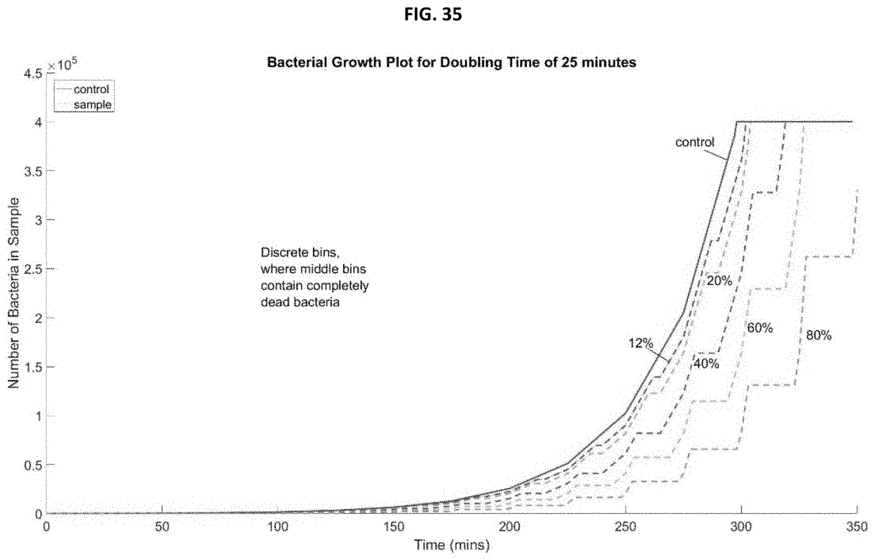

In modeling the growth of E. coli, the number of E. coli in a bin in the Bin Model is akin to the number of E. coli at a particular size. The resonant eradication is a function of size, so if a resonant frequency targets E. coli at it's average size then the central bins will be depopulated while the lower and higher numbered bins will remain unaffected. Assuming each bin of a 25 bin model has an equal number of bacteria, and the middle bin is eradicated, the lack of bacteria in a middle bin will result in no increase in bacteria at the time at which the eradicated bin would have replicated. This results in a step in the OD plot (FIG. 35). The length of the horizontal portion of the step indicates the number of bins or equivalently size range eradicated.

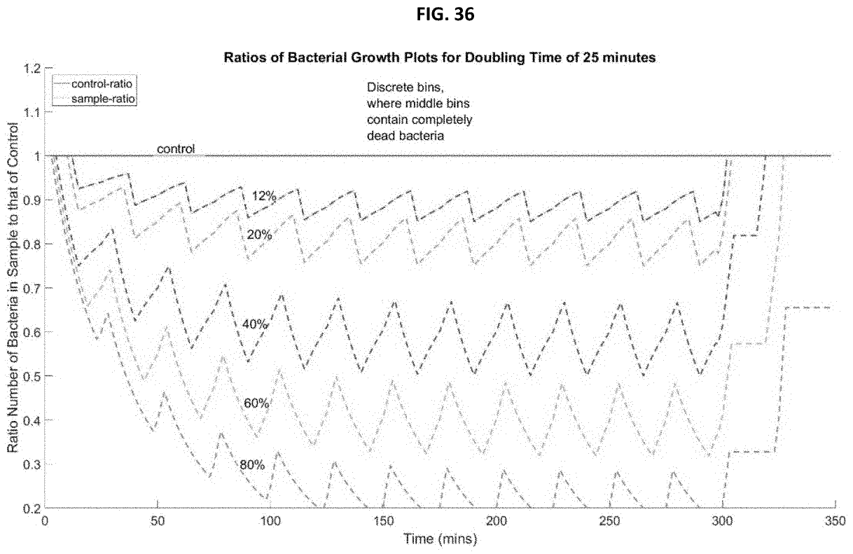

The equivalent OD-ratio plot (FIG. 36) shows a saw tooth feature. The vertical extent between valley and peak of the saw-tooth is related to the number of bins or equivalently size range affected during treatment.

Note that accurately determining relative (experimental vs control) bacterial growth levels depends upon separating out variations in initial conditions that may affect the growth value at any particular time. The model developed above provides a method for adjusting for variations in initial bacterial levels between samples and controls using ratios of OD derived bacterial growth curves. The unadjusted ratio curve values at the initial time can be used to adjust ratio curve values to accurately obtain eradication levels in the exponential stage of growth.

Accurately determining relative (experimental vs control) final growth levels depends on upon separating out variations in final nutrient levels between the experimental sample and control. The model additionally provides insight into final nutrient levels between controls and experimental samples for the unadjusted curves, and the relative live bacterial amount between the sample and control in the exponential phase of both using the ratio of the bacterial growth curves. Using these methods one can derive the effectiveness of various antibacterial methods that target specific stages of growth during binary fission. The model assumes accurate OD measurements of both the sample and control, and nearly identical temporal measurements.

Bacteria (e.g., E. coli) Oscillation Model

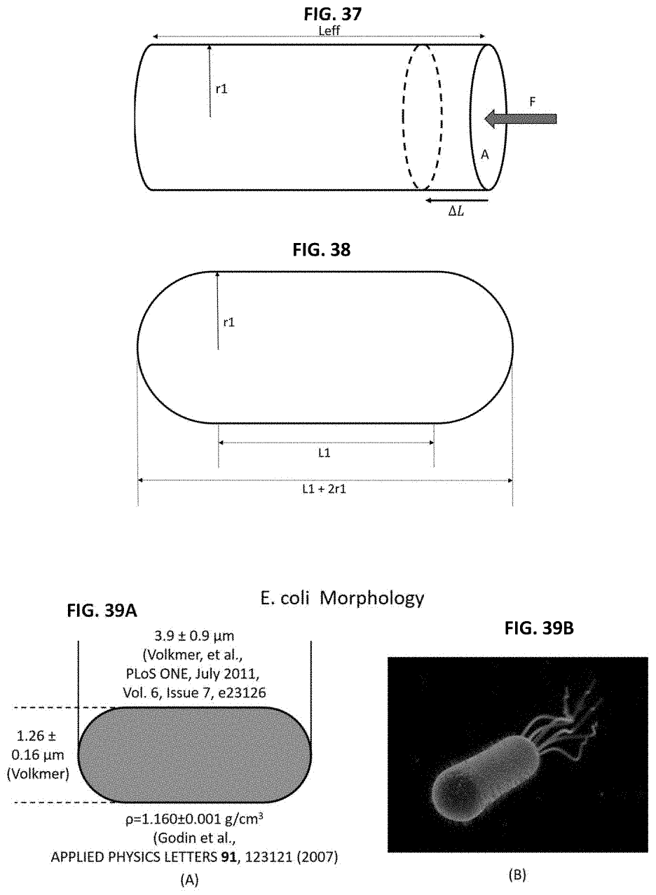

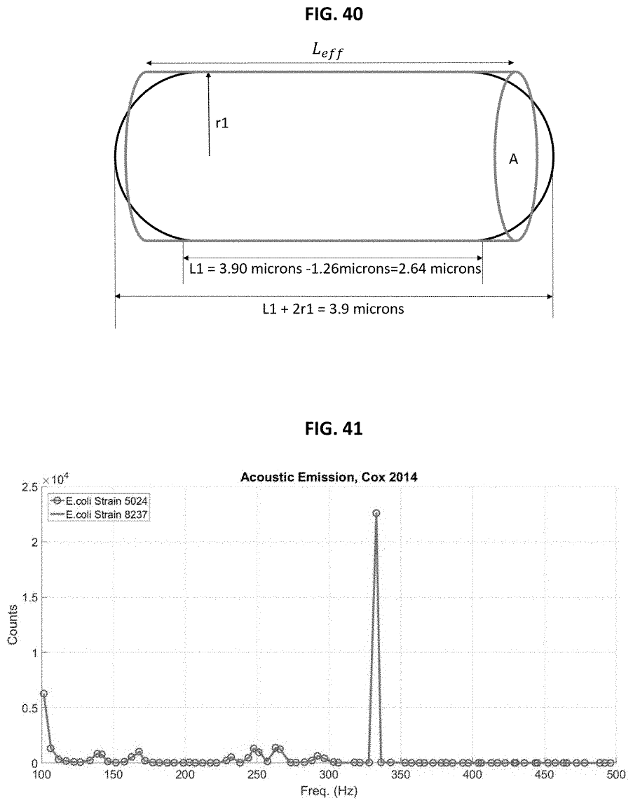

The current experimental setup limits pulse widths to be greater than the pathogen size (pulse condition 2) although exemplary embodiment include wavelengths less than pathogen size, in this case E. coli. Since E. coli changes primarily in one dimension during growth (lengthwise, see section 1.2.2), a rod oscillation model, similar to that used to derive equation 2.1.7, was used to determine resonant frequencies. Note that unlike the derivation of equation 2.1.7 which models a sphere into a rod, the E. coli model will model an oblong shaped E. coli as a rod. The rod oscillation model is a function of three basic parameters, effective equilibrium length (L_{eff}), density p, and Young's Modulus (E). FIG. 37 illustrates the basic model of linear deformation. The deformation of a rod of equilibrium length L_{eff} is dependent upon the elastic properties of the material of the rod. A measure of the elastic property can be expressed in terms of the Young's Modulus, E, previously described in equation 2.1.3:

$$E = \frac{( \frac{F}{A} )}{( \frac{Δ L}{{L}_{eff}} )} \qquad ( 2.1 .3 )$$

Rewriting equation 2.2.3 in the form of F=kΔL one obtains:

$$F = ( \frac{EA}{{L}_{eff}} ) Δ L \qquad ( 2.2 .15 )$$

The portion in the parenthesis can be viewed as an equivalent spring constant k, where the natural frequency can now be expressed as:

$$f = \frac{ω}{2 π} = \frac{1}{2 π} \sqrt{\frac{k}{m}} = \frac{1}{2 π} \sqrt{\frac{EA}{{mL}_{eff}}} \qquad ( 2.2 .16 )$$

The mass (m) is a function of the density and the volume, expressed as:

m=ρV=ρAL_{eff}=ρπr₁²L_{eff} (2.2.17)

Substituting (2.2.17) into (2.2.16) one obtains:

$$f = \frac{1}{2 π} \sqrt{\frac{EA}{ρ {AL}_{eff} {L}_{eff}}} = \frac{1}{2 π} ( \frac{1}{{L}_{eff}} ) \sqrt{\frac{E}{ρ}} \qquad ( 2.2 .18 )$$

In keeping with the resonant discussions of section 1, an integer fraction (1/n) of the natural/resonant frequency of the system can be expressed as:

$${f}_{n} = ( \frac{1}{n} ) \frac{1}{2 π} ( \frac{1}{{L}_{eff}} ) \sqrt{\frac{E}{ρ}} \qquad ( 2.2 .19 )$$

Frequencies can be calculated from equation 2.2.19, and are dependent upon three system dependent variables, L_{eff}, E and ρ. The goal becomes determining these three variables for a particular pathogen that can be modelled as a rod. E. coli has hemispherical caps at either end of the rod. Thus an equivalent L_{eff} must be determined. FIG. 38 illustrates the end capped geometry of E. coli, while FIG. 39A illustrates particular dimensions obtained from the literature, while FIG. 39B is an image of an E. coli. Since, E. coli expands in one dimension, this can be accomplished by equating the enclosed volumes. The volume of the shape of FIG. 38 can be expressed as:

V=4/3πr₁³+πr₁²L₁=πr₁²(4/3r₁+L₁) (2.2.20)

Matching volumes, one obtains:

V=πr₁²(4/3r₁+L₁)=πr₁²(L_{eff}) (2.2.21)

L_{eff} can be expressed as:

L_{eff}=(4/3r₁+L₁)=4/3(0.63 μm)+(2.64 μm)=3.48 μm (2.2.22)

Equation (2.2.19) can then be expressed as:

$${f}_{n} \approx ( \frac{1}{n} ) \frac{1}{2 π} ( \frac{1}{( \frac{4}{3} {r}_{1} + {L}_{1} )} ) \sqrt{\frac{E}{ρ}} \qquad ( 2.2 .23 )$$

Using the density expressed by Godin 2007 of ρ=1160 kg/m³ equation (2.2.23) can be expressed as:

$${f}_{n} = ( \frac{1}{n} ) \frac{1}{2 π} ( \frac{1}{3.48 \times {10}^{- 6} m} ) \sqrt{\frac{E ( \frac{kgm}{{s}^{2}} ) ( \frac{1}{{m}^{2}} )}{1160 kg \text{/} {m}^{3}}} = ( \frac{1}{n} ) \frac{1}{2 π} ( \frac{1}{3.48 \times {10}^{- 6} m} ) \sqrt{\frac{E ( \frac{1}{{s}^{2}} )}{1160 \text{/} {m}^{2}}} \qquad ( 2.2 .24 )$$

This can be summarized as:

$${f}_{n} = ( \frac{\sqrt{E ( in Pascals )}}{n} ) ( 1342.8017 ) Hz$$

Note that fₙ is a function of Young's Modulus, E. As shown in table 1 of section 1.2.2 the value of E for E. coli varies widely. For the particular strain used in proof of concept experiments, DH5α, the reported value of E can vary by as much as 2 MPa to 6 MPa (Cerf. 2009). To narrow down the value of E to use in a model, we turned to reported acoustic emission data for two strains of E. coli.

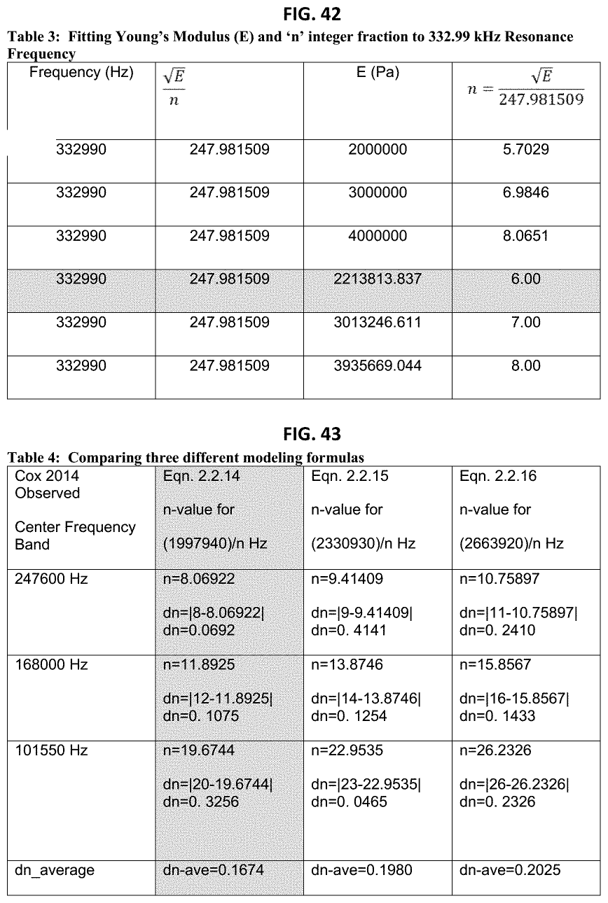

As discussed in 2014 Cox, in her Masters Thesis from the University of Kentucky, examined acoustic emissions produced from E. coli during the growth cycle, with the intent to identify specific frequency peaks that could be used as unique strain identifiers. She looked at two E. coli strains 5024 and 8237 and found unique spectral characteristics, but focused on frequencies less than about 50 kHz. Cox 2014 also reported higher frequency results but did not address their relevance. One frequency spike occurs identically for both E. coli strains. FIG. 41 illustrates the higher frequency data from Cox 2014. Using a value of 3±0.6 MPa for E and 332.99 kHz as one of the ‘nth’ value of resonance in equation 2.2.25, we can determine ‘n’ and E by matching 332.99 kHz and examine which value of E and ‘n’ more closely matches the other peaks reported by Cox 2014. Table 3 (FIG. 42) below provides various values of E and ‘n’ matched to provide an fₙ=332.99 kHz. Table 4 (FIG. 43) then provides predicted peaks compared to the center bandwidth frequencies reported by Cox 2014.

Ultimately we obtain:

$$\begin{matrix}{f}_{n} = ( \frac{1}{n} ) ( 1997940 ) Hz , \\ Derived from n = 6 and E = 2.213813 MPa , and & ( 2.2 .26 ) \\ {f}_{n} ( \frac{1}{n} ) ( 2330930 ) Hz , \\ Derived from n = 7 and E = 3.013246 MPa . & ( 2.2 .27 ) \\ {f}_{n} = ( \frac{1}{n} ) ( 2663920 ) Hz , \\ Derived from n = 8 and E = 3.935669 MPa . & ( 2.2 .28 )\end{matrix}$$

The shaded row of Table 3 indicates the selected values, discussed later with respect to Table 4, used to determine the frequencies used in the E. coli experiments. We can now compare the larger n-value predictions of equations 2.2.26, 2.2.27, and 2.1.28 to determine which best fits most of the peak frequencies seen. Table 4 compares the three predictions with observed values in Cox 2014 that are common for both strains examined.

Table 4 indicates the integer fraction ‘n’ predicted for each of the model formulas 2.2.26, 2.2.27, and 2.2.28. Three frequencies are matched that have similar peak values for each strains examined in Cox 2014, 247600 Hz, 168000 Hz, and 101550 Hz. Included in table 4 are the variations ‘dn’ between the predicted integer values and the closest integer. Each formula of equations 2.2.26, 2.2.27, and 2.2.28 first matched 332990 Hz. Note that the integer error increases the greater the n value for the predictions of equation 2.2.26. Also note that the average variation is lowest for the predictions of equation 2.2.26. Therefore, equation 2.2.26 is used to determine the frequencies use in the E. coli experiments (shaded column of Table 4).

As discussed in general, if a pathogen is deformed (e.g., 20%) or so the infectivity of the pathogen can be affected. The exposure time is defined as the time it takes to achieve the desired 20% deformation or another select % deformation (e.g., 0.5% to 300%). If we modify equation 2.2.3 using ΔL=0.2 L_{eff}, we can derive the net accumulated pressure needed as a function of elastic properties.

$$P = \frac{F}{A} = ( \frac{E}{{L}_{eff}} ) Δ L = ( \frac{E}{{L}_{eff}} ) 0.2 {L}_{eff} = 0.2 E \qquad ( 2.2 .29 )$$

To determine the time of exposure needed, we use ΔP as the increment of pressure increase per cycle of exposure, for example per pulse width. Note that ΔP can take into account damping, for example if 1 Pa is imparted each cycle then if damping is 30%, then ΔP=0.7 Pa. Exposure time, in number of cycles needed, can then be expressed generally as:

$${t}_{exp - cycles} = \frac{P}{Δ P} = 0.2 \frac{E}{μΔ P} \qquad ( 2.2 .30 )$$

Where μ is a damping factor from 0.0 to 100% damping.
The acoustic pulses are generally impingent upon samples at about 94 dB, or ΔP=1 Pa. Assuming the model of equation 2.2.26 is valid and E=2.213813 MPa, and a damping of 50%, we obtain exposure time needed of about

$${t}_{exp - cycles} = 0.2 \frac{2213813 Pa}{( 0.50 ) ( 1 Pa )} = 885525.2 cycles \qquad ( 2.2 .31 )$$

To obtain the time needed one must look at the frequency used, f.

$${t}_{exp - sec} = \frac{P}{Δ Pf} = 0.2 \frac{E}{μΔ Pf} = \frac{{t}_{exp - cycles}}{f ( Hz )} \qquad ( 2.2 .32 )$$

Using equation 2.2.32 with the parameters of equation 2.2.31 and a low frequency of 10000 Hz, one obtains:

$${t}_{exp - sec} = \frac{885525.2 cycles}{10000 Hz} = 88.5 sec \qquad ( 2.2 .33 )$$

All frequencies tested in experiments are greater than 10000 Hz so the exposure times at 50% damping is about 88.5 seconds. Tests vary from 120 seconds to 240 seconds.
Introduction E. coli Tests

The growth curves of the ampicillin resistant DH5α strain of E. coli and the surface integrity and mitochondrial metabolism of two host cells were examined after treatment with acoustic pulses. Acoustic emission frequencies observed in Cox 2014 and predicted frequencies using equation 2.2.26 were used and examined. The frequencies showing the largest bacterial effects were then tested on host cells (Vero and Human Bronchial) to test host cell surface disruption and mitochondrial metabolism disruption. The DH5α used in experiments was ampicillin resistant so that ampicillin could be added to all well samples. This way any contaminants would be eradicated and not affect results.

### Experimental Design/Procedures

As a proof of concept, acoustic experiments were performed on E. coli. In addition to testing growth reduction (e.g., eradication of a particular size), the effect of the acoustic pulses on cell walls and cell function were also tested, since the idea is to affect pathogens without affecting the host. Three basic experimental types were performed. First growth curves, as measured by Optical Density (OD) measurements at 600 nm, were obtained for various frequencies and exposure times to test effectiveness of growth reduction. Second, surface integrity studies were performed on two types of host cells (Vero and Human bronchial cells) using Trypan Blue techniques, to test cell rupture from the acoustic frequencies. Third, host cell function studies were performed using Mitochondrial metabolism (MTT) tests, to test cell function when exposed to the acoustic frequencies. Each experiment included controls and blanks. Controls, untreated bacteria and liquid broth (LB), were chosen to be located at various well locations throughout the 96-well experimental tray. Blanks were similarly chosen throughout the 96-well experimental trays, where blanks contained no bacteria only liquid broth (LB).

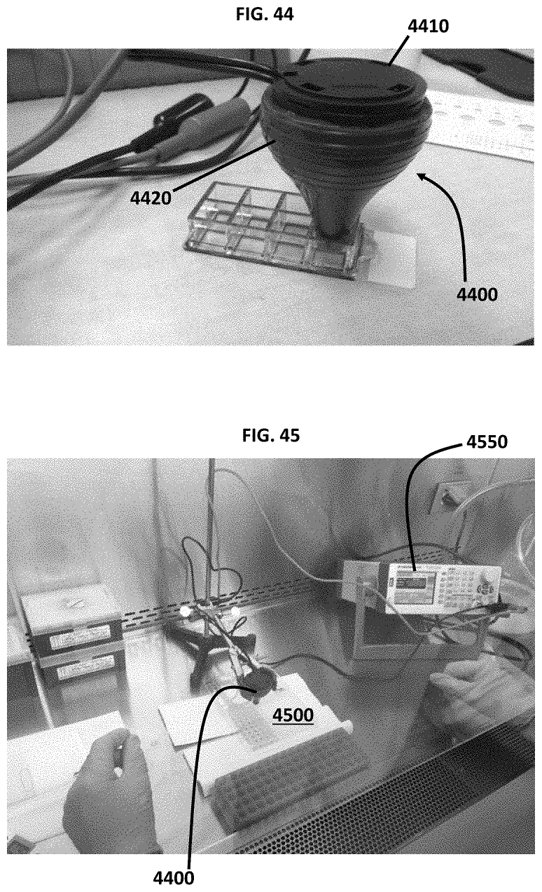

For the proof of concept an acoustic pulser 4400 (FIG. 44) was used in the bacterial studies. It was constructed from a speaker coil 4410 (black disk, with range 5 kHz to 25 kHz), connected to a curved waveguide adapter (green), driven by a function generator (4550, FIG. 45) at 10 to 20 volts peak-to-peak, in pulse mode for some studies and sine waves for others. Since many of the frequencies used were low frequency ultrasonic, difficult to hear by the experimenter, during operation an audible frequency was used first to verify operation of the pulser then switched to the test frequencies for the exposure period. At the end of each test the frequency of the pulser was again switched to an audible signal to verify operation. FIG. 45 is an image of the pulser positioned above a curved bottom 96-well tray. All tested frequencies are greater than 10 kHz and so (eqn. 2.2.33) indicates exposure times must be greater than 88.5 sec for 50% damping. Tests are performed at exposure times of 120 and 240 seconds.

$${t}_{exp - sec} = \frac{885525.2 cycles}{10000 Hz} = 88.5 sec \qquad ( 2.2 .33 )$$

FIG. 45 is an image of a typical experimental testing setup showing a 96-well tray with samples. The acoustic pulser is positioned above a particular well, not touching the lip of the well but coplanar with the top of a well. A timer keeps track of the exposure time at which time the frequency generator channel connected to the acoustic pulser is switched off.

The bacterial experiments are performed in a standard 96-well tray. The proof of concept is intended to acoustically treat bacteria on a surface, then resuspend them in food nutrient to compare the treated bacteria growth to controls where similarly situated bacteria, untreated, are likewise resuspended. Since the acoustic pulser is an air acoustic system and not a fluid ultrasonic system, the interface between air and bacterial suspensions fluid interface results in a large percentage, >90%, of the energy being reflected. To deliver the acoustic energy to the pathogen, in this case E. coli, the bacteria in LB growth medium suspension must first be evaporated, leaving behind only E. coli (simulated E. coli on a surface). In several experiments, ten (10) microliters of bacteria in LB are injected into the bottom of a well. The test tray (96-well) is then placed in a 37° C. incubator until all the fluid has been evaporated, except for a separate experiment where one test tray held dry samples and another the equivalent wet samples. When visual inspection determines that all of the test wells are evaporated, acoustic pulsing experiments are started. It is important to note that it takes anywhere from 1 to 2 hours to evaporate, and thus the bacteria will be undergoing binary fission during evaporation. This results in a variety of bacterial sizes when fully evaporated. As noted in previous discussions, a particular acoustic pulsing frequency is tailored for a particular size (e.g., L_{eff}), hence only a portion of the dry bacterial sample is expected to be affected during treatment. For 10 μl injected in a bottom of a u-well evaporation was generally within the acoustic pulser's spot size, which was assumed to be the inner diameter of the output orifice.

For several other experiments, 40 μl was injected into both u-bottom and flat bottom wells. 40 μl filled the entire curved bottom and a portion up the walls During evaporation of the u-bottom wells a substantial portion of the bacteria evaporated along the walls lying outside the treatment spot size. It was noted that the flat bottom wells evaporate from the center outward toward the outer edges of the bottom. It can be shown that the mobility of bacteria is greater than the evaporation rate of the fluid surface, so the bacteria can move outward with a higher density toward the edges, outside the acoustic pulser's spot size. Thus for several experiments it was expected that a majority of the bacteria would be untreated, more so for the flat bottom well.

After treatment the dry bacterial sample is re-suspended in 100 microliters of LB, where in and out pipetting of about 30-40 microliters is used to mix the suspension. The test tray is then examined over a period of time up to 24 hours, for bacterial growth, where any eradicated bacteria will not contribute to growth. The treated wells are compared to the control wells which underwent the same conditions (e.g., evaporation) but were not treated. Several results are discussed below.

After treatment, the 96-well tray is placed into an optical density measurement device. Optical density (OD) measures the temporally dependent dispersion of a particular wavelength of light through a sample. Each OD measurement is made with respect to designated blank wells, that contain the same nutrient bath (LB) amount but no bacteria. OD values increase in time as the number of bacteria in a sample increase. When a particular experiment is completed, the tray is then placed in the OD reader, which is set to shake the tray for about 35 seconds and then measure the OD, repeating the shaking-measurement step every hour for set period of time. Results are presented both as OD plots versus time and OD-ratio plots versus time.

### Surface Integrity (Trypan Blue) Procedure

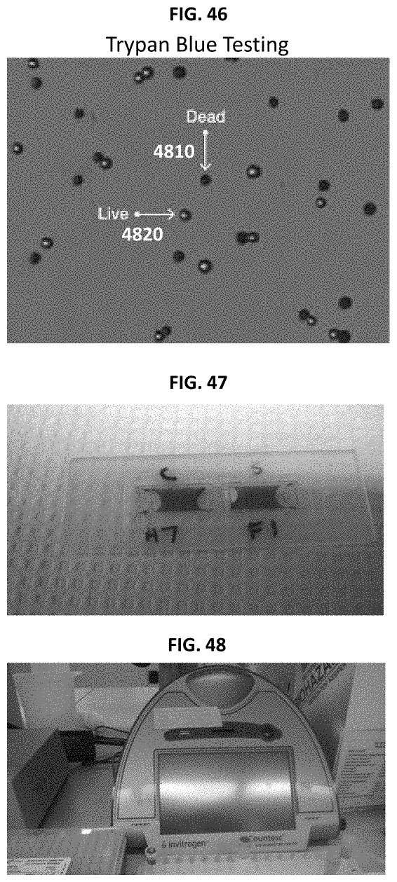

Trypan Blue is blue dye that is used in bacterial integrity studies to determine if the surface of a cell has been disrupted to a level that it allows the blue dye into the interior of the cell. It is a measure of cell surface integrity. Many methods exist that can eradicate bacteria (e.g., open flame) however the goal is to selectively disrupt bacterial functions while not harming host cells. For the bacterial studies two types of host cells were chosen, Vero (Monkey Kidney Cells) and Human Bronchial Cells. FIG. 46 illustrates a sample of dead (4610) and live cells (4620) in a trypan blue medium. In general, in a typical experiment, the first day the Vero cells were plated into a 96-well tray. The second day the cells were treated with selected acoustic frequencies that had proven effective in bacterial growth reduction. Then on the third day the treated cells were collected and placed in a sample slide, FIG. 47. The sample slide was inserted into a reader (FIG. 48) that counts the number of dead and live cells. The percentage of live to dead cells is compared to the control.

### Mitochondrial Metabolism Procedure

Trypan blue staining is not a sensitive method for determining internal cell function. Yellow MTT is a substance that is reduced to purple formazan in the mitochondria of healthy cells. The absorbance of MTT (Thiazolyl Blue Tetrazolium Bromide) by the mitochondria can be measured by the same OD measuring device used for bacterial growth, measuring the density of purple produced. Before measurement, the cell wall integrity must be compromised releasing the purple color into solution where the density of purple in solution is directly related to the number of cells with healthy mitochondria. The percentage of absorbance of MTT of a sample is compared to the control. FIG. 49A is an image of healthy Vero cells prior to MTT assay, while FIG. 49B shows the formation of purple formazan after MTT assay in a control (no treatment).

### Proof of Concept Results

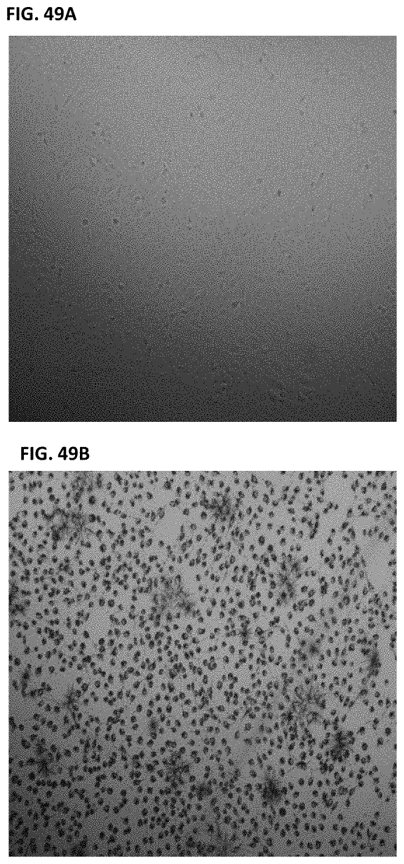

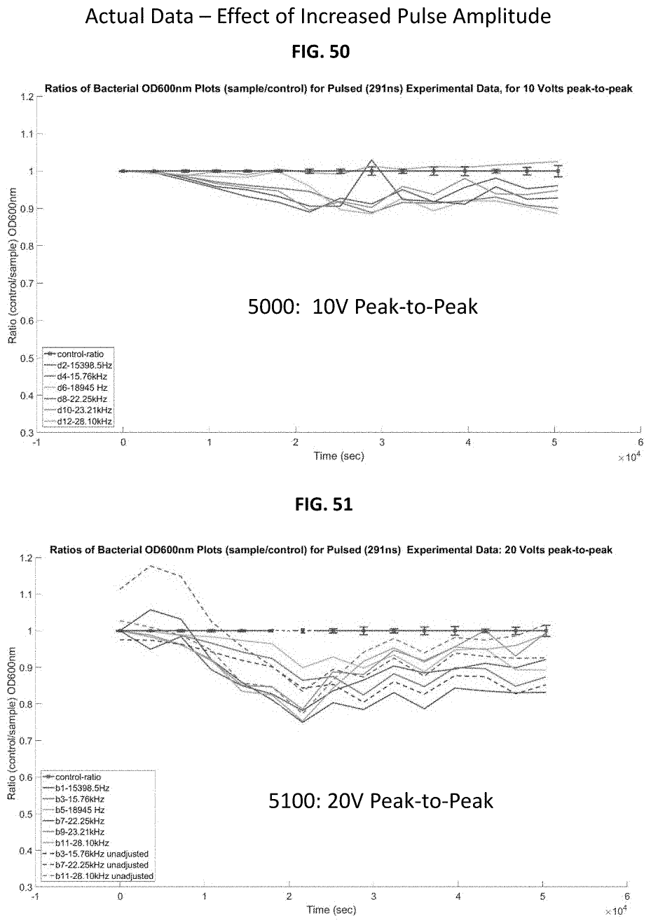

FIGS. 50 and 51 show experimental results with different peak-to-peak amplitude voltage on the function generator, keeping in mind that 20 Volts is about 94 dB acoustical level, for a control (red line) and several frequencies 15398.5 Hz, 15.76 kHz, 18945 Hz, 22.25 kHz, 23.2 kHz, and 28.1 kHz. FIG. 50, fora 10 Volt peak-to-peak (5000), bacterial eradication is roughly 10% (0.9 on vertical axis), while FIG. 51, for a 20 Volt peak-to-peak (5100) shows a bacterial eradication of about 20% (0.8 on vertical axis), for an exposure time of about 120 seconds. Hence a factor of two increase of eradication occurs with a factor of two increase in peak-to-peak voltage.

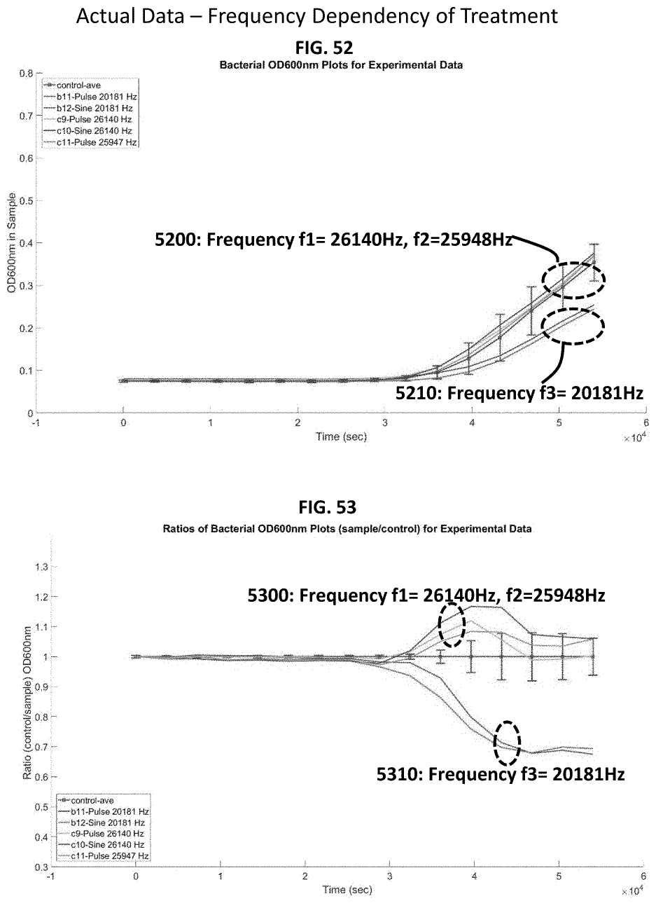

FIGS. 52 and 53 show experimental results of frequency dependency on bacterial growth reduction (e.g., eradication of some sizes). As can been seen, for the 20 Volt peak-to peak and 120 seconds exposure time, frequencies of 26140 Hz and 25947 Hz have little effect (5200, 5300), while a frequency of 20181 Hz eradicates roughly 30% (0.7 on vertical axis).

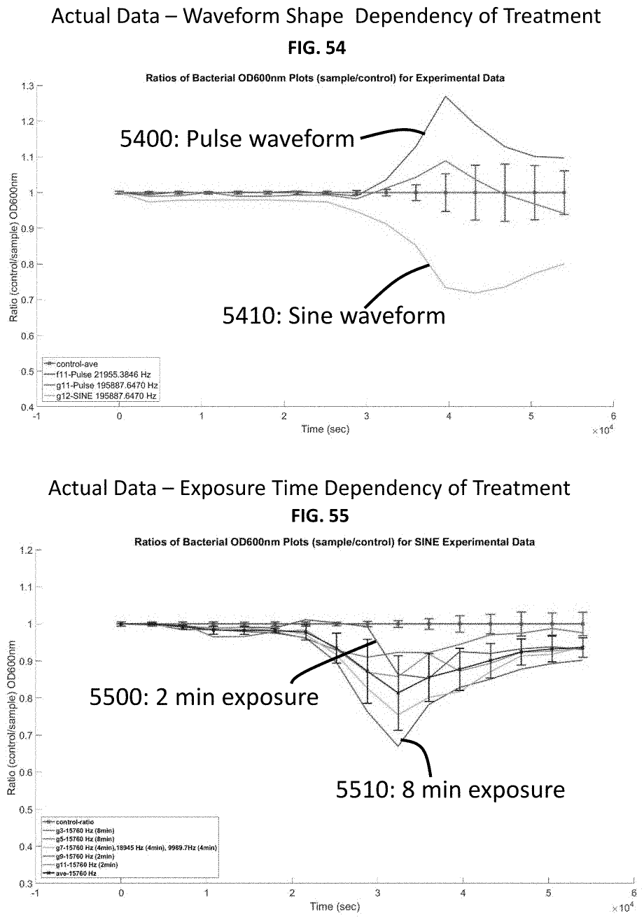

FIG. 54 shows experimental results of waveform dependency on bacterial growth reduction (e.g., eradication of some sizes). As can been seen, for the 20 Volt peak-to peak and 120 seconds exposure time, a pulse waveform 5400 (roughly 500 nm) has less effect than a sinusoidal waveform 5410. Note that this also applies to frequencies much less than resonant frequencies (roughly 1 million Hz for bacteria) since we are dealing with radial oscillations. For E. coli there is only axis expansion in binary fission replication. Therefore, an axial compression can manifest itself as a compression then expansion in the short cylindrical dimension and a compression and expansion in the longer axis. The compression and subsequent expansion in the long direction can result in premature binary fission prior to when the bacteria is ready for replication, resulting in both bacteria having compromised capsids or outer protein layers, rendering the bacteria sterile from that point onward or in a weaken state affecting infectivity.

FIG. 55 illustrates the effect on increased exposure with an 8 minute exposure time (5510) eradicating roughly 30% (0.7 on vertical axis) compared to a 2 minute exposure (5500) of 10% (0.9 on vertical axis). This is because of the various sizes of the bacteria during replication, and the longer the exposure time the more bacteria grow into the disruption frequency. If the exposure time matches the replication time (e.g., 25 min) then it would be expected that most of the bacteria would be disrupted.

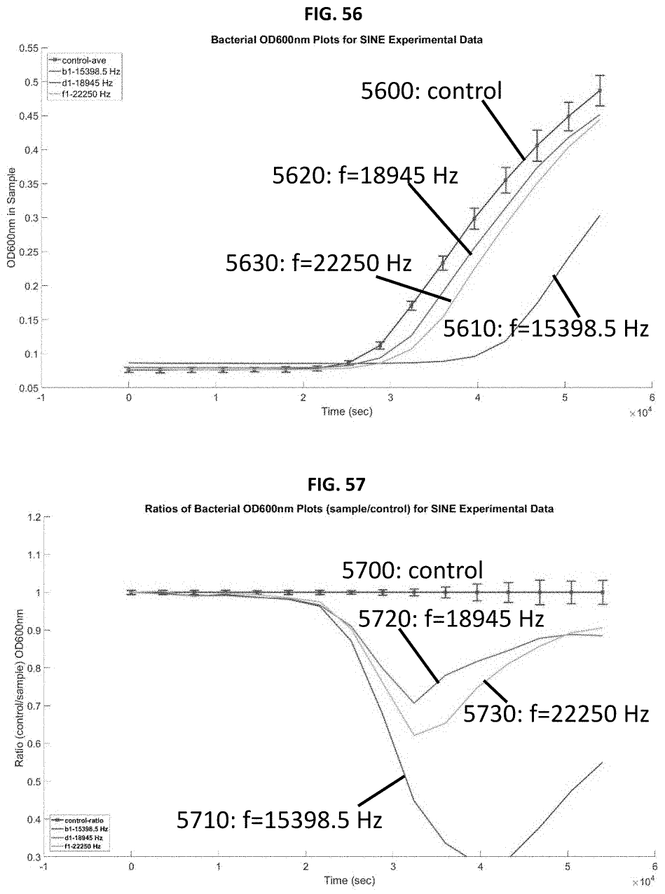

FIGS. 56 and 57 show experimental results of frequency dependency, using peak-to-peak amplitude voltage of 20 Volts, exposure times of 120 seconds, for the control (red line) and several samples exposed to several frequencies 15398.5 Hz (5610, 5710), 18945 Hz (5620, 5720), and 22.25 kHz (5630,5730). Frequency 188945 Hz (5620, 5720) eradicates roughly 25%, frequency 22250 Hz (5630,5730) roughly 35%, and frequency 15398.5 Hz (5610, 5710) roughly 27%. Thus, eradication effectiveness depends upon the frequency of treatment chosen, amplitude, waveform, and treatment duration. Note that the amplitude of the acoustic waves is not at the level that will destroy tissue initially, thus targeting at a particular frequency, wave shape, and amplitude over time results in targeted eradication of the pathogen leaving healthy tissue unharmed.

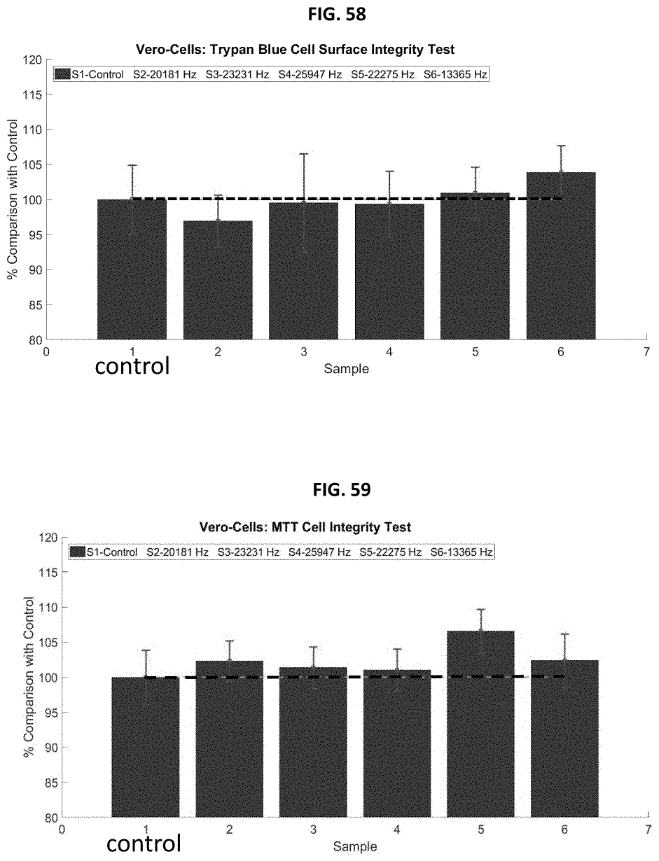

FIG. 58 illustrates the experimental data for the Trypan Blue surface tests for vero cells, at several frequencies also shown to cause bacterial eradication, indicating that within standard deviations there is little or no effect on the vero cells at the target frequencies used, showing that although several of these frequencies decrease bacterial growth, they would not harm vero cells. Suggesting that it is possible to target a pathogen without harming the membrane of host cells.

FIG. 59 shown the MTT cell integrity test for the same frequencies as used in FIG. 58, indicating no loss of mitochondrial function. In actually one frequency, 22275 Hz appeared to stimulate mitochondrial function. Thus, suggesting again that it is possible to target a pathogen without harming the mitochondrial function of the host cell.

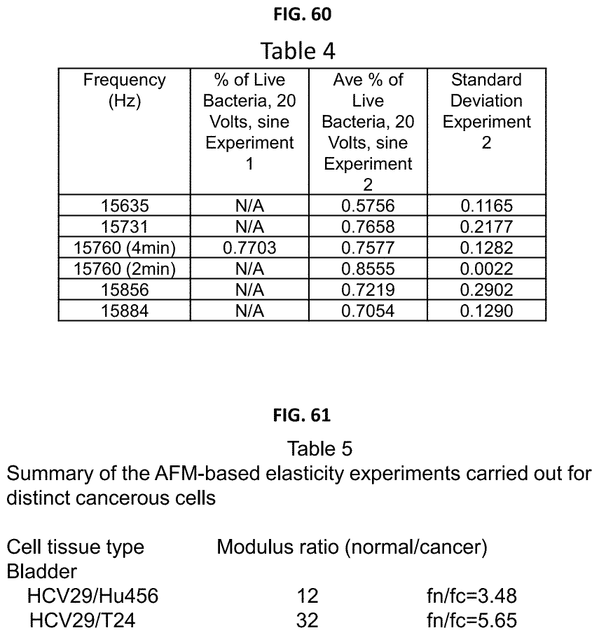

FIG. 60 illustrates table 4, which indicates the results of multiple experiments, at various frequencies, for exposure times of 120 sec (except where noted). For the frequencies listed up to about 42% eradication is achieved in 120 seconds, at roughly 94 dB (20V peak-to-peak) at 15635 Hz. Note discussion herein is not intended to limit the frequencies that are useful, each pathogen will have a resonant frequencies and the relevant integer fraction will need to be determined for each pathogen, so as to take into account damping, realistic exposure time, and harm to host cells.

Although the proof of concept examined E. coli bacteria in detail, as noted herein the processes are applicable to viruses, cancer and other non-normal cells. For example Table 5 (FIG. 61) indicates the elastic modulus difference (e.g., ratio of Youngs modulus E) of two types of bladder cancer cells, thus a difference in target frequencies between normal cells and cancerous cells. For example the Young's modulus E of HCV29/Hu456 cancer strain has normal to cancerous Young's modulus ratio of 12. If used in the resonance models above, a ratio of resonance frequency of normal cells (fn) to cancerous cells (fc) is 3.46. Suppose then for argument sake that the resonance of a normal cell is measured to be 1000 Hz=fn. Then the target frequency we could use is fc=1000 Hz/3.46 or 289 Hz. As noted above normal resonance frequencies are in MHz, hence the target frequency would be some integer fraction of the cancerous resonant frequency, with the added measure of making sure that that target frequency is also not some integer fraction of a normal cells resonant frequency.

### Additional Exemplary Embodiments

What follows are non-limiting descriptions of various acoustic generation and delivery methods, however in general if resonance frequency can be delivered then that frequency is used at amplitudes that will not harm normal cells, and if not obtainable then integer fractions of the resonance dependent upon the damping. Exemplary embodiments are directed to a device to generate or receive acoustic waves, that can be used as an acoustic source (e.g., speaker) and acoustic microphone (e.g., microphone). In particular exemplary embodiments discussed utilize fluid-based or laser-based generated acoustic waves to generate high frequency acoustic waves to generate acoustic resonance to deactivate/destroy viruses (MHz to GHz). Note that similar exemplary embodiments can generate hearing acoustic and ultrasonic frequencies (e.g., 10 Hz-50 kHz) and can be used as speakers and microphones. Additionally, traditional transducers and/or coil speakers can be used if the frequencies desired are in the audio range (50 Hz to 10000 Hz) as well as ultrasonics that can be used in fluids and air even at extended ranges (10,000 Hz to GHz).

At least one exemplary embodiment is directed to generating a high frequency acoustic source to set up acoustic integer fractions of resonance in live pathogens. In addition to acoustic sources, acoustic detectors of various types can be used, provided the sensitivity is enough to detect acoustic emissions above the noise floor.

Processes, techniques, apparatus, and materials as known by one of ordinary skill in the art may not be discussed in detail but are intended to be part of the enabling description where appropriate. For example, specific materials may not be listed for achieving each of the targeted properties discussed, however one of ordinary skill would be able, without undo experimentation, to determine the materials needed given the enabling disclosure herein. Additionally, various techniques, formulas, in acoustical physics and photoacoustics is assumed. Thus, the contents of “Photoacoustic Imaging and Spectroscopy” edited by Lihong V. Wang, CRC Press, Optical Science and Engineering #144 is incorporated by reference in its entirety, as is the “fundamentals of physical acoustics” by David T. Blackstock, ISBN 0-471-31979-1 which is also incorporated by reference in its entirety.

Notice that similar reference numerals and letters refer to similar items in the following figures, and thus once an item is defined in one figure, it may not be discussed or further defined in the following figures. Processes, techniques, apparatus, and materials as known by one of ordinary skill in the relevant art may not be discussed in detail but are intended to be part of the enabling description where appropriate.

FerroFluids (FF) and Magnetorheological Fluids (MRF): Ferrofluids (also refrred to as magnetoresponsive fluids (MR)) can be composed of nanoscale particles (diameter usually 10 nanometers or less) of magnetite, hematite or some other compound containing iron. This is small enough for thermal agitation to disperse them evenly within a carrier fluid, and for them to contribute to the overall magnetic response of the fluid. Ferrofluids can include tiny iron particles covered with a liquid coating, also surfactant that are then added to water or oil, which gives them their liquid properties.

Ferrofluids are colloidal suspensions—materials with properties of more than one state of matter. In this case, the two states of matter are the solid metal and liquid it is in this ability to change phases with the application of a magnetic field allows them to be used as seals, lubricants, and may open up further applications in future nanoelectromechanical systems. In at least one embodiment a sample of ferrofluid can be mixed with various other fluids (e.g., water, mineral oil, alcohol) to acquire various desired properties. For example, when mixed with water and a magnetic field is applied the ferrofluid will separate from the water pushing the water in the opposite direction from the ferrofluid. Such a system can be used as a pump to move fluid from one side of a bladder to another, or even into a separate region, for example where the water can react to an agent when the ferrofluid would not. Another example of a benefit to mixing is to vary the viscosity of the fluid. If the ferrofluid is mixed with mineral oil, the net fluid is less viscous and more easily moved, while remaining mixed when a magnetic field is applied. If the net fluid is in a reservoir chamber one can move the fluid into a different chamber by application of a magnetic field. Note that the discussion above applies equally well for an ER fluid where electric fields are applied instead of magnetic fields.

True ferrofluids are stable. This means that the solid particles do not agglomerate or phase separate even in extremely strong magnetic fields. However, the surfactant tends to break down over time (a few years), and eventually the nano-particles will agglomerate, and they will separate out and no longer contribute to the fluid's magnetic response. The term magnetorheological fluid (MRF) refers to liquids similar to ferrofluids (FF) that solidify in the presence of a magnetic field. Magnetorheological fluids have micrometer scale magnetic particles that are one to three orders of magnitude larger than those of ferrofluids. The specific temperature required varies depending on the specific compounds used for the nano-particles.

The surfactants used to coat the nanoparticles include, but are not limited to: oleic acid; tetramethylammonium hydroxide; citric acid; soy lecithin These surfactants prevent the nanoparticles from clumping together, ensuring that the particles do not form aggregates that become too heavy to be held in suspension by Brownian motion. The magnetic particles in an ideal ferrofluid do not settle out, even when exposed to a strong magnetic, or gravitational field. Steric repulsion then prevents agglomeration of the particles. While surfactants are useful in prolonging the settling rate in ferrofluids, they also prove detrimental to the fluid's magnetic properties (specifically, the fluid's magnetic saturation). The addition of surfactants (or any other foreign particles) decreases the packing density of the ferroparticles while in its activated state, thus decreasing the fluids on-state viscosity, resulting in a “softer” activated fluid. While the on-state viscosity (the “hardness” of the activated fluid) is less of a concern for some ferrofluid applications, it is a primary fluid property for the majority of their commercial and industrial applications and therefore a compromise must be met when considering on-state viscosity versus the settling rate of a ferrofluid.

Ferrofluids in general comprise a colloidal suspension of very finely-divided magnetic particles dispersed in a liquid carrier, such as water or other organic liquids to include, but not limited to: liquid hydrocarbons, fluorocarbons, silicones, organic esters and diesters, and other stable inert liquids of the desired properties and viscosities. Ferrofluids of the type prepared and described in U.S. Pat. No. 3,917,538, issued Nov. 4, 1975, hereby incorporated by reference in its entirety, may be employed. The ferrofluid is selected to have a desired viscous-dampening viscosity in the field; for example, viscosities at 25.degree. C of 100 to 5000 cps at 50 to 1000 gauss saturation magnetization of the ferrofluid such as a liquid ferrofluid having a viscosity of about 500 to 1500 cps and a magnetic saturation of 200 to 600 gauss. The magnetic material employed may be magnetic material made from materials of the Alnico group, rare earth cobalt, or other materials providing a magnetic field, but typically comprises permanent magnetic material. Where the permanent magnetic material is used as the seismic mass, it is axially polarized in the housing made of nonferromagnetic material, such as aluminum, zinc, plastic, etc., and the magnet creates a magnetic-force field which equally distributes the enclosed ferrofluid in the annular volume of the housing and on the planar faces of the housing walls.

The proposed method utilizes a physical principle well known in the physical sciences called resonance. When an engineering object is designed and built, resonance must be taken into account to avoid catastrophic build up of vibrations that occur at the resonant frequency of the object. The proposed method would gradually build up vibration energy in a virus by impacting the virus with acoustic waves at the virus's integer fraction of the resonant frequency (if the resonant frequency can not be reached with equipment), which is a function of the size, density and geometry of the virus. The method, applied for a period of time, would tear apart the targeted virus in a patient's body without interjecting any anti-viral chemicals into the patient's system. The remaining portions of the virus could be used by the immune system of the patient to develop antibodies.

In general a virus can range in diameter from 20 nm to about 300 nm. If the resonant frequency is solely based upon viral size the needed acoustic frequency would be in the GHz range, as discussed above in detail. The actual viral resonant frequencies are unknown. A simplified air bubble in water model provides a resonant frequency of about 65.6 MHz for a dimension of about 100 nm, much smaller than reported by molecular models.

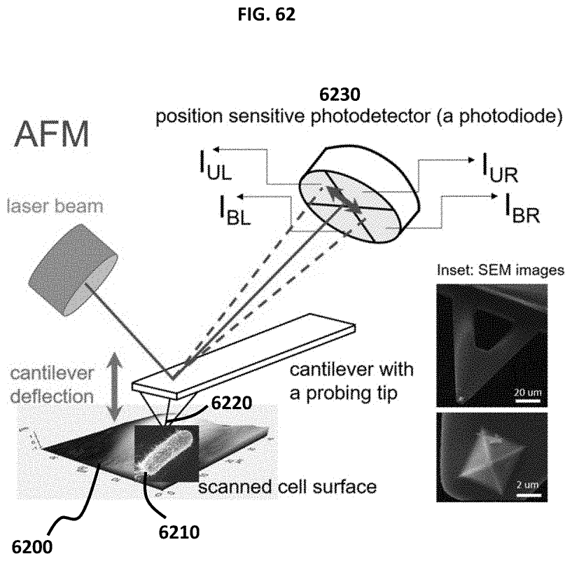

Determining the resonance frequency to serve as a basis for figuring out which integer fraction of the resonant frequency to use, is often the problem. FIG. 62 illustrates a method using an atomic force microscope in obtaining the natural or resonance frequency of a pathogen in at least one exemplary embodiment. Another is to send an acoustic pulse into a suspension with the pathogen in it to stimulate a resonance, and listen using ultrasonic pickup to the acoustics generated (emitted by the pulse stimulated pathogen). This gives both the resonant frequency and the damping over time. Both of these factors (resonant frequency and damping coefficient) can then be used to determine the integer fraction of the resonance frequency to use as a target (treatment) frequency. The damping coefficient is used, as discussed above, to determine exposure time needed.

The method illustrated in FIG. 62, uses oscillation of the platform 6200, upon which rests the pathogen 6210 (virus, bacteria, cancerous cell, abnormal cell) of interest or a normal cell (e.g., to obtain reference frequencies to avoid in treatment). The photodetector 6230 picks up the vibration as spatial oscillation, whose amplitude is related to the size of the oscillation passing through the pathogen 6210. When the oscillation of the platform reaches an integer fraction of the natural resonance of the pathogen 6210, more of the oscillation energy is passed through the pathogen 6210 to the cantilever tip 6220, and is translated as a larger spatial oscillation on the photodetector 6230. Sweeping of the platform 6200 frequencies can be used to map the pathogens 6210 integer and ultimately fundamental resonant frequency (n=1).

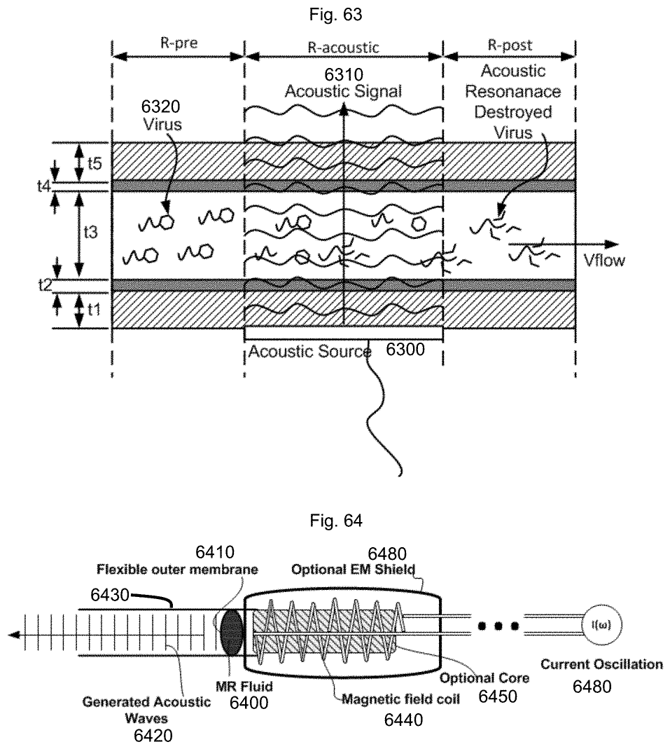

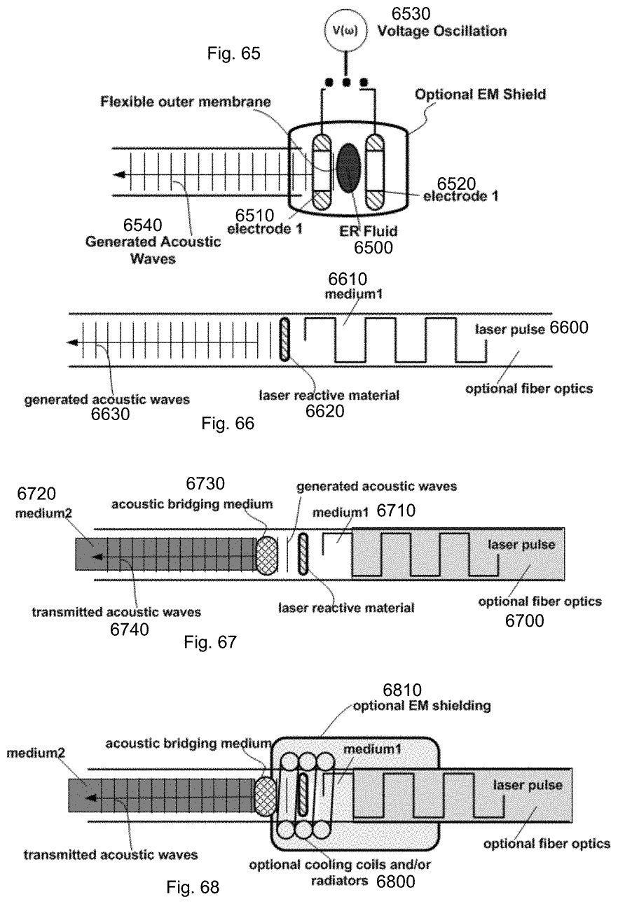

FIG. 63 illustrates a simplified model of an acoustic wave penetrating various levels of materials (e.g., skin, vascular walls, blood), where the transmitted acoustic waves set up resonances in the viruses that eventually ruptures or adversely effects the capsid or outer proteins or the virus (e.g., exceeds the elastic limit). In the simplified view an acoustic source 6300 (e.g., as described in FIG. 64, FIG. 65, FIG. 66, FIG. 67, and FIG. 68) generates an acoustic wave to a virally infected region for a period of time necessary to eradicate at least a target % (e.g., 5%, 10%, 25%, 50%, 75%) of the target virus per cubic mm of the virally infected region. The virally affected region is the region in which the acoustic wave travels that contains the target viruses. For example, acoustic source 6300 generates the acoustic signal 6310, which passes through a portion of a region that contains the target virus 6320. The region through which the acoustic signal passes can include multiple levels (e.g., skin tissue, vascular walls, blood). Each level can affect the acoustic energy (e.g., damping, dispersion) of the acoustic signal. The acoustic signal can be any type of periodic wave (e.g. sine wave, comb functions, ramp functions, cosine waves) that can be used to set up resonance frequencies in the targeted viruses, bacteria, or cancer (pathogen). In the example discussed we refer to viruses but exemplary embodiments can be used for any pathogen (which includes foreign bodies, abnormal cells, and cancer). Since each target virus has a unique size, and composition each virus will have a unique resonance frequency. The acoustic wave 6310 can be tuned to the viral resonant frequency to eradicate just the target virus.

FIGS. 64 through 70 illustrate various unique ways to generate acoustic signals. FIG. 64 illustrates at least one exemplary embodiment of generating acoustic waves using an oscillating fluid medium 6400 (e.g., a magneto responsive fluid (MR) or any other field responsive fluid). For example, a field responsive medium 6400 (e.g., MR and/or ER Fluid) enclosed with at least one side having a flexible membrane 6410 (e.g., silicone, urethane, rubber) can be oscillated in response to an oscillating field (e.g., magnetic and/or electric field). In the non-limiting example illustrated in FIG. 64, the field responsive medium 6400 oscillates generating an acoustic wave 6420, which can be guided by a waveguide 6430. The optional waveguide 6430 can be chosen so that the propagation modes are at the desired frequencies. A field oscillating device (e.g., magnetic coil 6440 and optionally core 6450 (e.g., ferrous)) is configured to generate an oscillating field at a target frequency moving the field responsive medium 6400 at the target frequency, where movement of the field responsive medium generates acoustic waves (e.g., 6420) at about the target frequency. For example if the field oscillating device includes a coil 6440, oscillation of a magnetic field can be accomplished via an oscillating current source 6460 with optional additional circuitry. An optional shield 6470 (e.g., EM, mu metal, microwave) can encompass the field oscillating device to avoid any harmful waves reaching a user or patient.

In the alternative an impinging pressure wave will move the field responsive medium 6400 within a background field generated by the field oscillating device generating a current that oscillates in response to the movement of the field responsive medium 6400. Thus, the system can additionally or alternatively act as a microphone.

FIG. 65 illustrates at least one exemplary embodiment of generating acoustic waves using an oscillating field responsive medium (e.g., an electro responsive fluid (ER)). In the non-limiting example illustrated the field responsive medium 6500 responds to electric fields generated by a varying potential difference between a first electrode 6510 and a second electrode 6520. The varying potential can be generated by an oscillating voltage source 6530 is coupled to the first electrode 6510 and the second electrode 6520 with optional additional circuitry. As the voltage is changes the field responsive medium moves, generating acoustic waves 6540. Alternatively, impinging pressure waves can oscillate the field responsive medium 6500, varying the capacitance between the first (6510) and second (6520) electrodes which can, as also discussed with respect to FIG. 64, be converted into electronic signals representing the pressure waves. Thus at least one exemplary embodiment using an electric field responsive medium and/or magnetic field responsive medium, can be used as a microphone. Note that microphone is a general term not intended to limit the frequency of the acoustic or pressure waves detected. Note also a cooling coil and/or radiator can be used to cool the system (e.g., the field generating device) of the field responsive medium systems (e.g., FIG. 64 and FIG. 65), in addition to the laser systems described (e.g., FIG. 66, FIG. 67, and FIG. 68).

FIG. 66 illustrates at least one exemplary embodiment of generating acoustic waves using photo acoustics. An acoustic wave can also be generated by a pulsed laser generating a laser pulse 6600, travelling through a first medium 6610 (e.g., air), that impinges upon a laser reactive material 6620, that oscillates in response to the laser pulse 6000. For example, a material that expands (e.g., thermal expansion) and contracts at a frequency corresponding to the pulse frequency. The expansion and contraction generates an acoustic wave 6630. The laser can optionally be delivered to the laser reactive material 6620 via a fiber optic cable 6700. Note that in the alternative an impinging pressure field on a layer (e.g., the laser reactive material 66200, or in this case even a material that reflects at least a portion of the incident laser pulse) will oscillate the layer. The oscillation of the layer can be detected by ranging the laser pulses (e.g., detecting a Doppler shift) thus acting as a laser reflective based microphone. The microphone can be as small as a fiber optic cable where the layer can oscillate in response to a pressure wave.

FIG. 67 illustrates at least one exemplary embodiment of generating acoustic waves using photo acoustics in one medium 6710 and coupling the generated acoustic waves into a second medium 6720. An acoustic bridging medium 6730 can be used to isolate the first medium 6710 (e.g., air) from the second medium 6720 (e.g., water) and still transmit (6740) at least a portion of the generated 6740 or received acoustic wave between the two mediums (6710 and 6720).

FIG. 68 illustrates at least one exemplary embodiment of generating acoustic waves, with optional cooling coils and/or radiators 6800 and optional shielding 6810. In some cases the frequencies generated may heat materials and the materials may need to be cooled. Additionally, certain frequencies can generate EM wavelengths that might be harmful (e.g., burning) and thus shielding can be used.

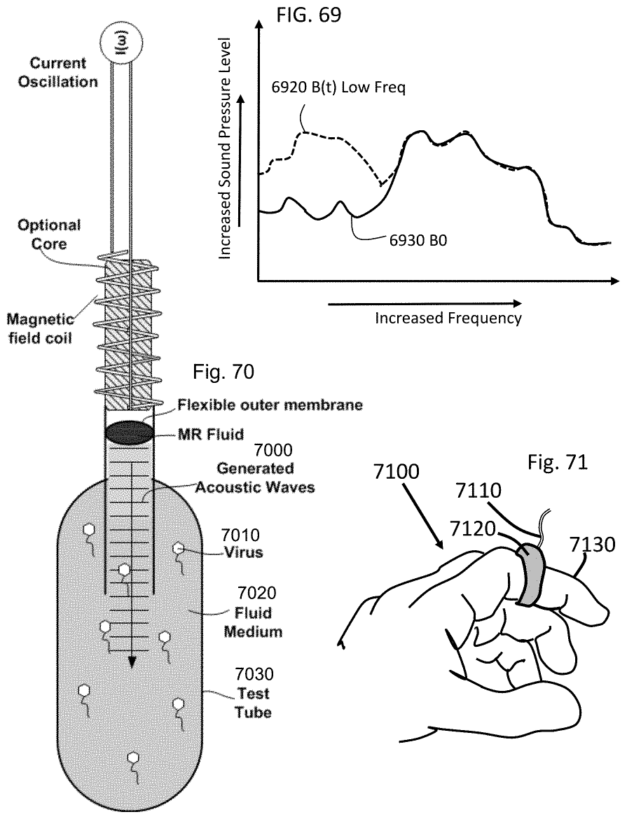

FIG. 69 illustrates data from a proof of concept experiment generating an acoustic wave in from an oscillating MR fluid bladder by changing the magnetic field at low frequencies. A background acoustic field 6930 is measured with a stable magnetic field BO. A field oscillating device began a low frequency broadband oscillation stimulating oscillation of a responsive field medium which generated the acoustic waves 6920 at the varying magnetic field frequencies B(t). In the limited example the increase in sound pressure level was about 30 dB.

FIG. 70 illustrates an example of using an acoustic generated acoustic wave 7000 to sterilize a pathogen 7010 infected medium 7020 in a test tube 7030.

FIG. 71 illustrates at least one exemplary embodiment 7100 of coupling an acoustic wave (e.g. optionally via acoustic waveguide, if transducers and circuitry can not fit within ring) to a ring device 7120 on a finger 7130 to input the acoustic waves into a patient's body for analysis and treatment. Note that eradication of the entire targeted virus is not necessary in some cases where the decreased level of live virus can provide the body's immune system to eradicate or control the remaining level of viruses.

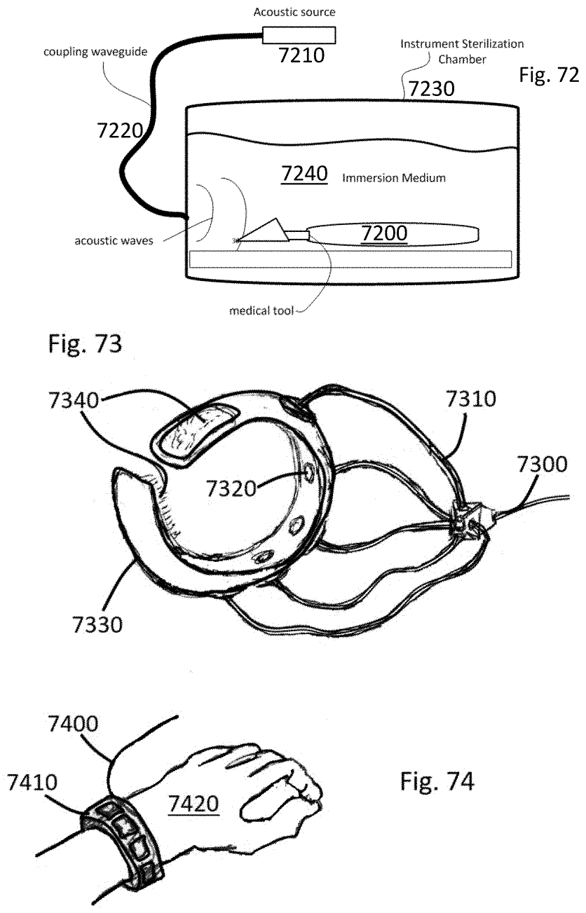

FIG. 72 illustrates at least one exemplary embodiment of coupling an acoustic wave (e.g., via acoustic waveguide) to an instrument 7200 (e.g., medical surgical instrument) to sterilize (eradicate a portion of the population of the targeted virus attached to) the instrument 7200. In this non limiting example, the acoustic source 7210 generates an acoustic wave that is coupled 7220 (e.g., waveguide) to an instrument sterilization chamber 7230. The acoustic waves are tuned for a targeted viruses resonance frequency. The acoustic waves traveled through an immersion medium (e.g., water) 7240 to the instrument 7200 (e.g., surgical device), setting up a vibration in the viruses that eradicate a certain number of the viruses within and exposure time. Note alternatively the instrument 7200 itself can be vibrated (e.g., via phonons or a mechanical vibrator) at the viral target frequency to disrupt and/or destroy targeted viruses. In such a system the acoustic source can be a vibration system coupled to the instrument 7200.

FIG. 73 illustrates at least one exemplary embodiment of coupling an acoustic wave (e.g. via acoustic waveguide) to a limb wrap device, that can be wrapped around a limb to input the acoustic waves into the patient body. For example, an acoustic source can be coupled via a waveguide 7300 that can be coupled to multiple waveguides 7310, where the multiple waveguides deliver an acoustic signal to various locations 7320 that touch the patient's skin. The various locations can be part of a wrap 7330 that can be secured around a limb using a fastening method 7340 (e.g. Velcro™).

FIG. 74 illustrates at least one exemplary embodiment of coupling 7400 an acoustic wave (e.g. via acoustic waveguide) to a wrist wrap device 7410, that can be wrapped around a wrist 7420 to input the acoustic waves into the patient body.

Note that additional exemplary embodiments can include playing a subject in a water or fluid bath, and emitting ultrasonics in the bath at the target frequencies. The advantage of this method is that the two mediums are similar (water/fluid and body tissue) and hence acoustic reflectivity is decreased at the surface.

As discussed above one can vary current and/or voltage to generate acoustical energy. Note also that if a steady field is imposed, then when sound impinges upon a field responsive medium (liquid, gas, solid) an induced current and/or voltage is generated. The induced current and/or voltage can be converted by known methods to pick up sound thus the systems described can also in certain configurations act as microphones.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions of the relevant exemplary embodiments. For example, if words such as “orthogonal”, “perpendicular” are used the intended meaning is “substantially orthogonal” and “substantially perpendicular” respectively. Additionally although specific numbers may be quoted in the claims, it is intended that a number close to the one stated is also within the intended scope, i.e. any stated number (e.g., 20 mils) should be interpreted to be “about” the value of the stated number (e.g., about 20 mils). Note also that the term “determine” can also be used to refer to a table or reference to obtain a value.

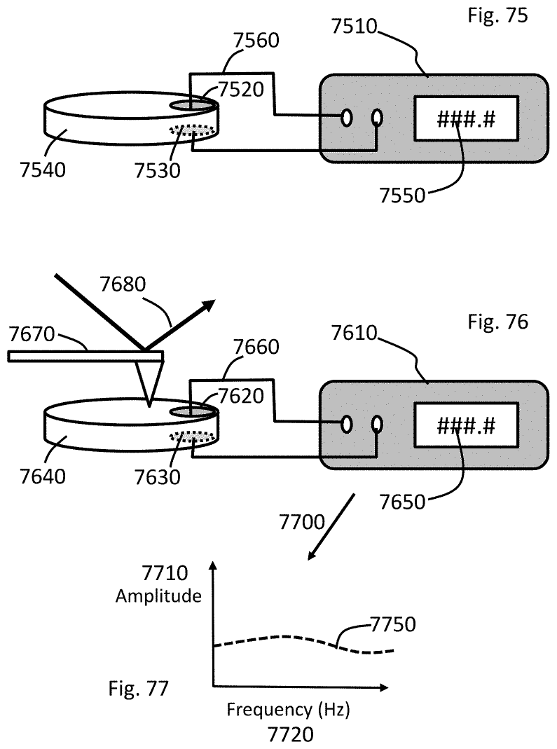

FIG. 75 illustrates a method of creating an oscillating AFM platform. A substrate 7540 (e.g., piezoelectric) can vibrate so that when a pathogen is placed on the platform, if the vibration is at the resonant frequency of the pathogen the vibrational energy will more easily passed to an AFM probe. An example of the platform, can be formed using a piezoelectric 7540, with electrodes 7520, 7530 placed on either side of the substrate 7540. An oscillating voltage is applied to electrodes 7520, 7530 using function/wave generator 7510 via contact wires 7560, setting the frequency 7550.

FIGS. 76 and 77 illustrate an AFM needle on an oscillating platform and the measurement taken. FIG. 76 illustrates a vibrating platform 7640, having an oscillatory voltage across electrodes 7620 and 7630, through wires 7660, generated by function generator 7610 set to a value 7650. The amplitude 7710 versus frequency 7720 generated is shown in the plot 7750 of FIG. 77. The platform oscillation is picked up by probe head 7670 by movement of the head picked up by the laser 7680.

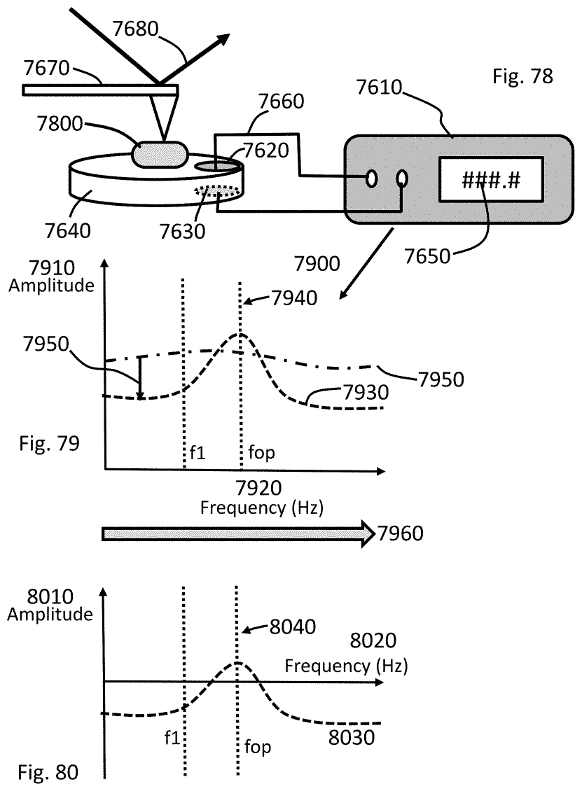

FIG. 78 illustrates a method to obtain the resonance frequency or integer fraction of the resonance of a pathogen 7800 directly. The frequency generator 7610 sweeps through various values 7650 imposed by contact wires 7660 to electrodes 7620 and 7630 oscillating platforms 7640. When the frequency of the platform more closely matches the resonance or integer fraction of the resonance of the pathogen the energy is more efficiently transferred to the probe head 7670 as measured by the probe laser 7680.

FIG. 79 illustrates the result 7900 of sweeping 7960 AFM platform oscillation frequency and measuring the amplitude that makes it to the AFM probe. The plot 7900 plots amplitude 7910 versus frequency 7920, where 7950 is the plot of the oscillating platform with the AFM without the pathogen. Once the pathogen 7800 is between the head 7670 and oscillating platform 7640. The vibrations from the platform 7640 are damped 7950 unless the platform oscillations are at the resonance frequency or integer fraction resonance of the pathogen 7800 then the amplitude 7940 peaks in the plot 7930.

FIG. 80 illustrates a plot 8030 of the modified result of sweeping AFM platform oscillation frequency 8020 versus amplitude 8010 that makes it to the AFM probe, subtracting the plot 7950 from the pathogen sweep plot 7930. A peak frequency 8040 identifies a resonant or integer fraction of the resonance of the pathogen 7800.

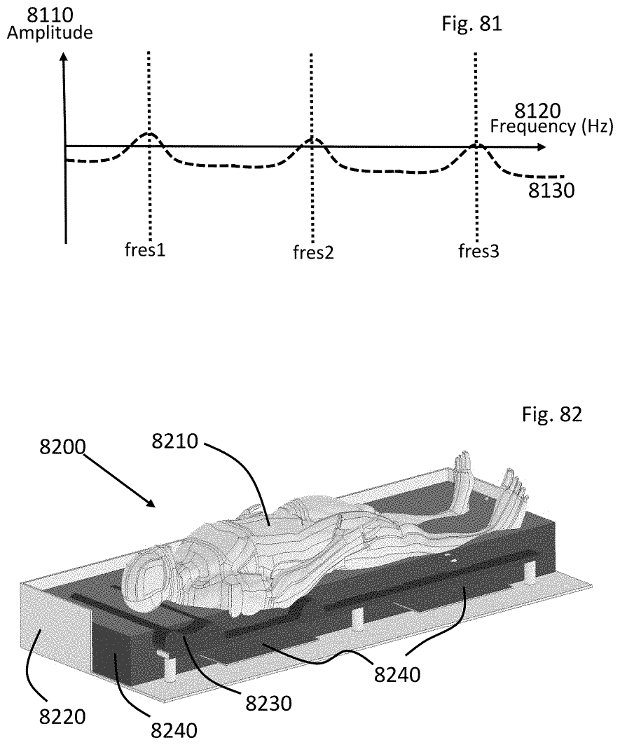

FIG. 81 illustrates a plot 8130 of a modified result of sweeping AFM platform oscillation frequency 8120 versus amplitude 8110 that makes it to the AFM probe across multiple integer fractions of the resonance frequency. The highest frequency with no further peaks should be the resonance frequency while the lower frequencies are integer fractions thereof.

FIG. 82 illustrates a water method for delivering the acoustic frequencies necessary for body 8210 treatment in a prone position 8200. The body 8210 to be treated lays upon a bed support 8230. The treatment bed 8200, includes a container 8220 that holds fluid 8240 which acts as an acoustic conductor that conducts the acoustic waves from the acoustic source 8240 to the body 8210.

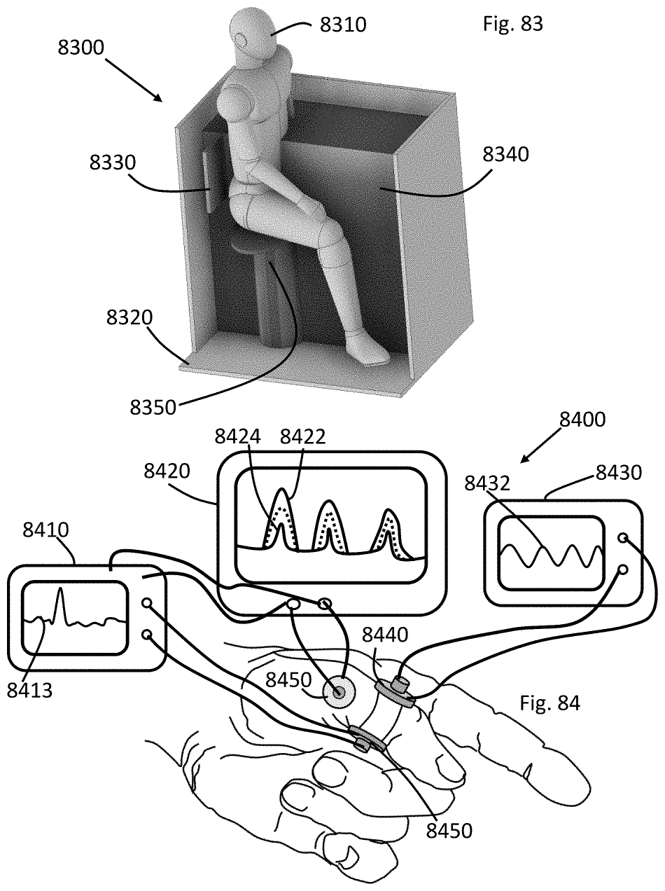

FIG. 83 illustrates a water method for delivering the acoustic frequencies necessary for body treatment in a sitting position 8300. The body 8310 to be treated sits upon a bench 8350. The treatment chamber 8300, includes a container 8320 that holds fluid 8340 which acts as an acoustic conductor that conducts the acoustic waves from the acoustic source 8330 to the body 8310.

FIG. 84 illustrates a finger method of analysis and treatment. A set up 8400 assembled to fit on a finger analyzes any abnormalities from sensor 8450, isolating the relevant frequencies (8422 and 8424) as displayed 8420. A function generator 8410 generates a treatment frequency 8413 that matches resonance or an integer fraction of the resonance that is sent to an acoustic generator 8450. A sensor 8440 can also be added to monitor the frequency generated by the generator 8450 with a measured signal 8432 monitored and shown on display 8430 with the measured signal fed back to the acoustic generator to keep the generated frequency at the chosen frequency.

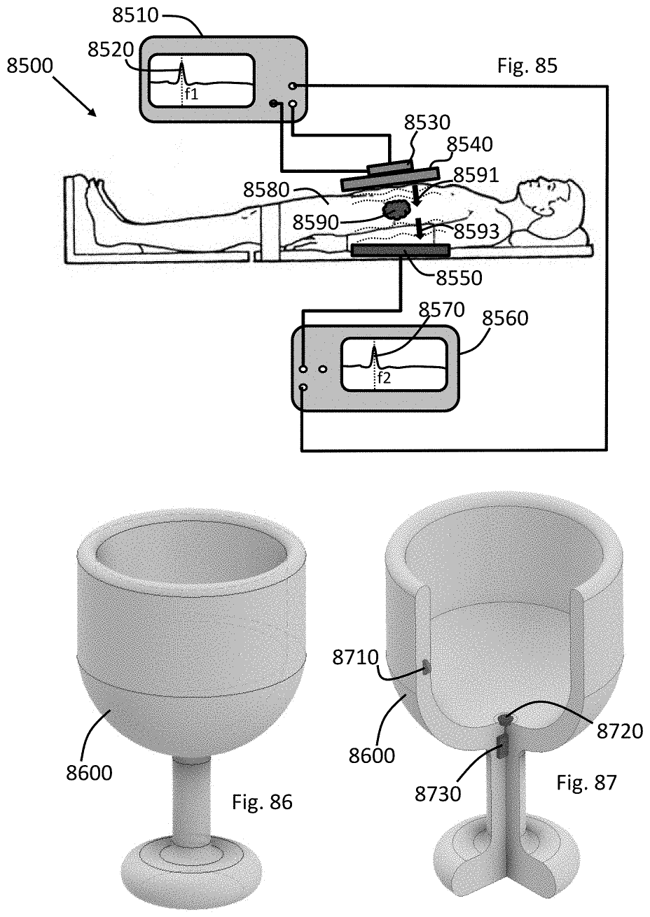

FIG. 85 illustrates a prone method 8500 of analysis and treatment. A signal generator 8510 sets the acoustic frequency f1 and waveform to be generated by acoustic generator 8530 with an optional body coupling interface 8540 (e.g., a gel layer). The acoustic waves 8591 generated by acoustic source 8530 travels within the body 8580. The acoustic waves 8591 impinges upon the cancer 8590 or other pathogen (e.g., bacteria, virus, cyst). The pathogen has a characteristic resonance frequency different than healthy tissue. The acoustic waves or pulses 8591 can be at the resonant frequency or about (e.g., within 20%) of an integer fraction of the pathogen's resonant frequency. The exposure time will depend upon the amplitude (chosen so as to not damage healthy tissue/cells) and the dampening of the acoustic wave/pulse in the body. A sensor 8550 detects a remnant of the acoustic wave/pulse 8593. The sensor 8550 is connected to signal detector 8560 which measures the frequency f2. The idea is to provide feedback to the signal generator 8510 to maintain the acoustic source at the desired frequency f1. System 8500 is a non-limiting example of a treatment setup. The basic principles can be used for various out of body sterilization, such as water sterilization. FIG. 86 illustrates a cup that sterilizes a fluid and optionally identifies a pathogen to sterilize. In some cases only targeted sterilization may be desired.

FIG. 86 illustrates a cup 8600 incorporating analysis and treatment method for pathogen eradication. FIG. 87 illustrates a cross-section of the cup 8600 incorporating analysis and treatment method for pathogen eradication. The cup can include a processor 8730, an acoustic source and/or detector 8720 at the base, and optionally a second acoustic source and/or detector 8710. When fluid is inserted into the cup, source 8720 can impart a pulse, then switched to a detector 8720 or 8710 can be used as a detector to listen for any resonances that match stored values of pathogens in memory connected to the processor 8730. Additionally, or alternatively the acoustic frequency peaks measured after initial pulsing can be compared with stored profiles for a clean fluid. The frequency peaks that are different between the measured and stored values for clean fluid can be used to target any pathogen impurities, Acoustic source 8720 can then emit frequency, amplitude and waveform for a treatment period (which may be different depending upon pathogen). Then a follow up pulse can be emitted to detect if any pathogen remains that resonates.

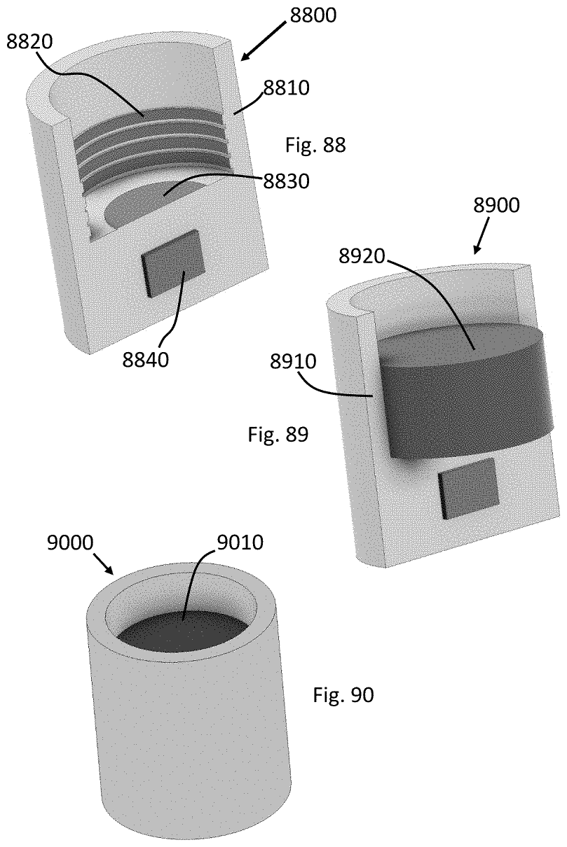

FIG. 88 illustrates a cross-section of an analysis and treatment sampling cup 8800. A vessel (e.g., cup) 8810 can have a processor (e.g., DSP) connected to acoustic source and/or detector 8830, and circumferential acoustic source and/or detectors 8820. FIG. 89 illustrates 8900 the cross-section 8910 of an analysis and treatment sampling cup 8800 with an inserted sample 8920. FIG. 90 illustrates 9000 the analysis and treatment sampling cup 8800 with the inserted sample 9010.

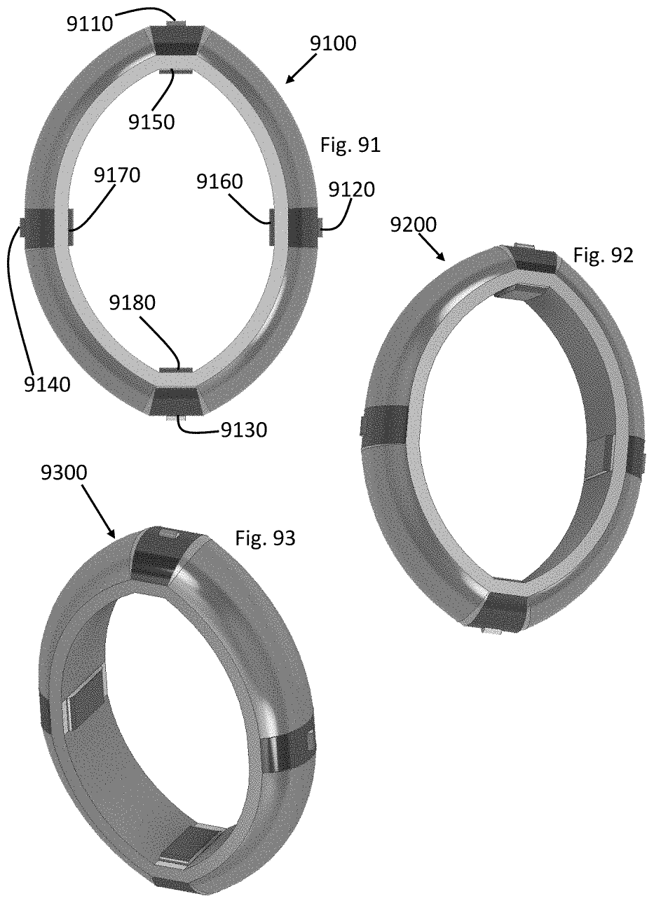

FIGS. 91, 92, and 93 illustrate several views of a biometric/treatment/analysis sensor bracelet 9100 that can additionally analyze and treat pathogens, and/or monitor a wearer biometric value. For example, sensors 9150, 9160, 9170 and 9180 can be acoustic signal generators and detectors, that deliver a treatment acoustic signal to a wearer's arm. For example, suppose a limb is damaged such that the doctor is worried about the wound going septic. The bracelet 9100 can be placed away from the wound closer to the body so that any infection traveling back toward the heart can be treated and rendered sterile reducing the chance of a wound going septic. Additionally, the bracelet can be used to monitor the user's biometrics and detect abnormalities including pathogen detection. The detection characteristics (e.g. frequency peaks) can be used then during treatment to affect pathogen spreading in tissue or blood stream through the bracelet 9100. Indicator elements (e.g., LED, treatment ongoing blinking LEDs, analysis certain color light) 9110, 9120, 9130, and 9140 can also be user interactive elements (e.g., buttons) to control or initiate operation (e.g., start analysis, start treatment). Some of the sensors 9150, 9160, 9170 and 9180 can be biometric measurement devices (Note the bracelet can contain a DSP, battery, a communication module to communication the biometrics wirelessly) such as transducer, infrared detector, capacitance sensor, optical sensor, ultrasonic sensor, RF sensor, thermal sensor, pressure sensor, and MEMs sensors. Note that vibrating systems, such as that used in automatic toothbrushes, bone conduction vibration sources can be used as the acoustic/vibrational source. The important aspect is to create a vibration in the medium that reaches the pathogen, it can be via acoustics or via direct vibration, for example a transducer for acoustic, or a piezoelectric oscillated in an electric field for vibrations in contact with the patient or vessel holding a sample.

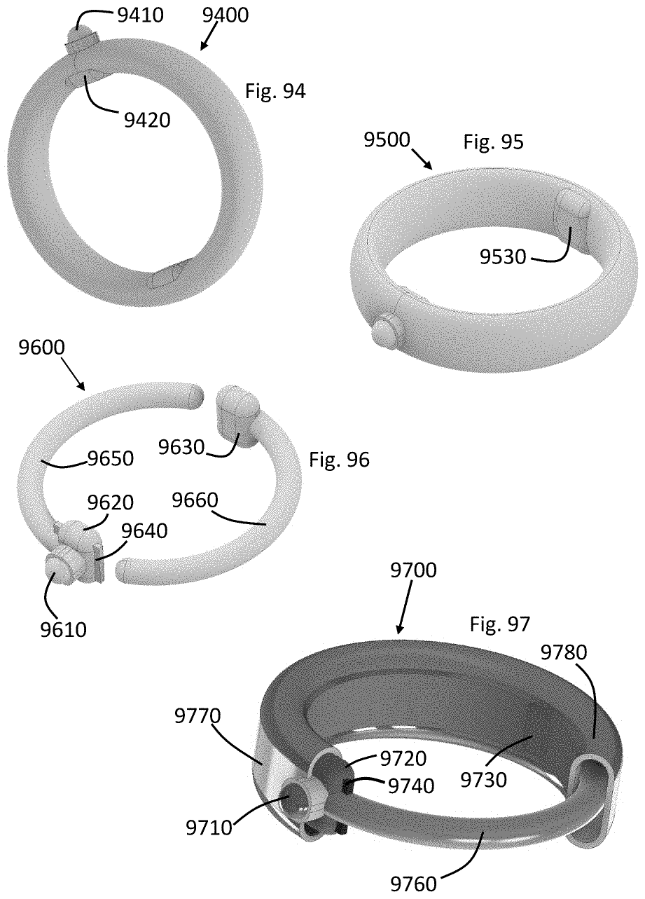

FIGS. 94, 95, 96, and 97 illustrate several views of a biometric sensor ring 9400, 9500, and 9700 that can additionally analyze and treat pathogens. Biometric sensor ring 9400 can include an indicator or a user interactive element 9410. Element 9420 can be a sensor, a vibration or acoustic source/detector or a biometric sensor. FIG. 95 illustrates a different view showing sensor 9530 which monitors or delivers acoustics/vibrations to a finger. FIG. 96 illustrates inner electronics 9600 of a biosensor ring. The inner electronics can include battery 9660, capacitor 9650, sensors 9620 and 9630, and a processor/DSP 9640, and interactive element 9610. FIG. 97 illustrates a cross section 9700 of sensor ring with housing 9780, solar cell recharging band 9770, sensor/source 9720, DSP/processor 9740, battery 9760, contact portion of sensor/source 9730. Element 9710 can be a user interactive element or an indicator light.

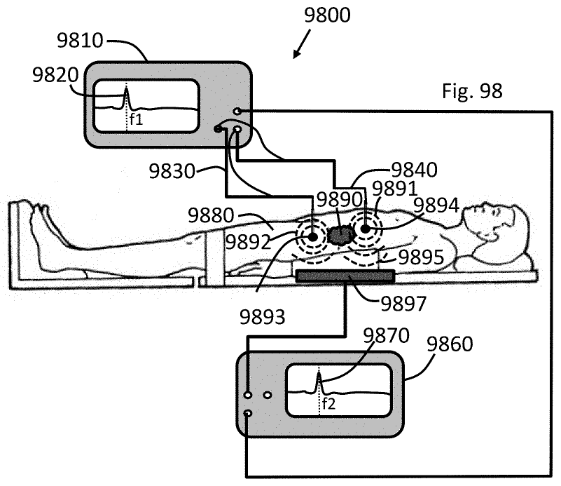

FIG. 98 illustrates a method of analysis and treatment using inserted acoustic detectors and emitters. FIG. 98 illustrates a prone method 9800 of analysis and treatment. A signal generator 9810 sets the acoustic frequency f1 and waveform to be generated by inserted acoustic generators 9893 and 9894. The acoustic waves 9892 and 9891 generated by acoustic sources 9892 and 9894 respectively travels within the body 9880. The acoustic waves 9891 and 9892 impinges upon the cancer 9890 or other pathogen (e.g., bacteria, virus, cyst). The pathogen has a characteristic resonance frequency different than healthy tissue. The acoustic waves or pulses 9891 and 9892 can be at the resonant frequency or about (e.g., within 20%) of an integer fraction of the pathogen's resonant frequency. The exposure time will depend upon the amplitude (chosen so as to not damage healthy tissue/cells) and the dampening of the acoustic wave/pulse in the body. The inserted acoustic/vibration sources 9892 and 9894 are inserted near the cancer site to deliver the vibrations/acoustic waves with less body damping. A sensor 9897 detects a remnant of the acoustic wave/pulse 9895. The sensor 9897 is connected to signal detector 9860 which measures the frequency f2. The idea is to provide feedback to the signal generator 9810 to maintain the acoustic source at the desired frequency f1. System 9800 is a non-limiting example of a treatment setup.

Although the discussion herein discusses viruses, bacteria, cancer and other abnormal cells and objects, the processes herein can be used to target selective release of medicine. For example, medicine pills (e.g., with medicine inside a shell) can be targeted for release of medicine at a particular location. For example, suppose a cancer fighting agent needs to be delivered to a particular region. Nano or microsized pills can be injected into blood upstream of the cancer cells. Monitoring the location of the pills by their acoustic signature with respect to ultrasound not at their resonance, one can wait until the pills are at the desired location. Then a targeted vibration/sound can be emitted to disrupt the pills to deliver the medicine or treatment locally.

Although often virus and bacteria are discussed in treatment examples, all pathogens can be treated, with any abnormal cell (e.g., cancer) or foreign object considered a pathogen in this specification. Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

What is claimed is:

1. A method of pathogen suppression comprising:
determine the fundamental acoustic or vibrational resonant frequency of a pathogen;
determine the integer fraction ‘n’ of the resonant frequency to use for a target frequency of a therapy vibrational wave;
determine the waveform of the vibrational wave;
determine the minimal exposure time to expose the pathogen to the vibrational wave; and
exposing the pathogen to the vibrational wave for a time that is at least as long as the minimal exposure time.

2. The method according to claim 1, where the step of determining the fundamental acoustic or vibrational resonant frequency is to use an atomic force microscope (AFM), oscillating the AFM platform, with the pathogen on the platform, and mapping the pickup amplitudes by the AFM sensor as a function of frequency to obtain a set of integer fraction resonant frequency peaks that are used to obtain the resonant frequency.

3. The method according to claim 1, where the resonant frequency is obtained by placing the pathogen in a suspension, pinging the suspension with an acoustic pulse, measuring the emitted acoustic spectrum from the pathogen to determine the resonant frequency.

4. The method according to claim 1, further including the step of:
determining the damping information of the pathogen at the target frequency.

5. The method according to claim 4, further including the step of:
determining the minimal amplitude of the vibrational wave needed based upon the damping information.

6. The method according to claim 5, where the minimal exposure time is determined by using an amplitude, a frequency, and damping information related to the target frequency vibrational wave to deform the pathogen by a threshold value after the pathogen is exposed to the vibrational wave for the minimal exposure time.

7. The method according to claim 1, where the pathogen is at least one of a virus, a bacteria, or a cancer cell.

8. The method of claim of claim 1 where the vibrational wave is an acoustic wave and has a frequency within 10% of the integer fraction ‘n’ of the resonant frequency.

9. The method according to claim 1, where “n” is a positive integer greater than zero.
